(19) United States
(12) Patent Application Publication
(10) Pub. No.: US 2017/0252442 A1
(43) Pub. Date: Sep. 7, 2017

(54) PHOTOHEXER COMPOUNDS AND PHARMACEUTICAL COMPOSITION AND USE THEREOF

(71) Applicants: Qicheng FANG, Beijing (CN); Wei FANG, Cape Coral, FL (US); RAINBOW PHARMATECH LLC, Cape Coral, FL (US)

(72) Inventors: Qicheng FANG, Beijing (CN); Wei FANG, Cape Coral, FL (US)

(21) Appl. No.: 15/506,700

(22) PCT Filed: Feb. 27, 2015
(86) PCT No.: PCT/CN2015/073344

§ 371 Date: Feb. 24, 2017

(30) Foreign Application Priority Data
Aug. 27, 2014 (CN) 201410427833.6

(51) Int. Cl. A61K 41/00; A61N 5/06; A61K 31/437; C07D 487/14

(52) U.S. Cl. CPC A61K 41/0076; A61K 41/0042; C07D 487/14; A61N 5/06; A61K 31/437; A61K 2121/00; A61K 2123/00

(57) ABSTRACT

The present invention relates to photohexer compounds and a pharmaceutical composition and use thereof. Specifically, the present invention relates to 2(4)-(1-hexyloxy-ethyl)-6,7-bispropionate-1,3,5,8-tetramethyl-4(2)-vinylpor-phyrin and their analogues, which are water soluble, have stable properties, can be used as a photosensitizer, and are suitable for the diagnosis and treatment of malignant tumors, precancerous lesions or benign lesions. The invention also relates to pharmaceutical compositions comprising such novel compounds, their use and preparation methods.

### TECHNICAL FIELD

The present invention relates to novel compounds used in photodynamic therapy (PDT). Specifically, the invention relates to 2(4)-(1-hexyloxyethyl)-6,7-bis-propionate-1,3,5,8-tetramethyl-4(2)-vinylporphyrin and their analogues, which are water soluble, have stable properties, can be used as a photosensitizer, and are suitable for the diagnosis and treatment of malignant tumors and precancerous lesions or benign lesions. The invention also relates to pharmaceutical compositions comprising such novel compounds, the use of such compounds and methods for preparing such compounds.

### BACKGROUND TECHNOLOGY

Cancer has become one of the main diseases that seriously threaten human health in recent years. Global cancer deaths statistics in 2008 from World Health Organization (WHO) show that around the world each year about 11 million new cases of cancer occur. New cancer cases in the Asia Pacific region accounted for 45% of the new global cancer cases, up to 7 million patients died of cancer each year. In recent 30 years, the world cancer incidence increased by 3%-5% each year, wherein ¾ of new cases occurred in the newly industrialized and developing countries. The number of cancer deaths in the Asia Pacific region accounted for about half of all cancer deaths in the world. China's population accounts for ⅕ of the world's total population, and 3.12 million new cases of cancer occur each year in China, the number of deaths due to cancer is 2.7 million.

The report of twenty-second Asia Pacific Cancer Congress held in Tianjin on October 31 to Nov. 2, 2013 pointed out that the annual new cancer patients in China accounted for more than 20% of the global new cancer patients. The deaths due to 8 types of cancer i.e. lung cancer, liver cancer, stomach cancer, esophageal cancer, colorectal cancer, cervical cancer, breast cancer and nasopharyngeal cancer account for more than 80% of the total number of cancer deaths. At present, cancer has become the first killer among the diseases in China. Due to serious side effects of traditional treatment methods such as radiotherapy and chemotherapy, as well as urgent needs for the targeted cancer therapy and individual therapy, photodynamic therapy (PDT) is one of the new strategy to change the traditional concept of cancer treatment. The above-mentioned 8 cancers, accounting for more than 80% of the total cancer deaths in China, are all indications of this therapy. The photodynamic therapy can treat the above-mentioned cancers which are discovered early, alleviate the suffering of patients with middle-stage and late-stage cancer, and improve their quality of life.

The basic principle of photodynamic therapy is as follows: a photosensitizer is injected in vivo to tumor patients, tumor sites are irradiated by a specific wavelength of laser for a certain residence time by means of selective photosensitizer uptake and retention of tumor cells. In the participation of molecular oxygen in the biological tissue, a strong photochemical reaction can be induced, resulting in very active ROS (reactive oxygen species) components such as singlet-state oxygen, free radicals and the like, to promote oxidation of a variety of biological macromolecules such as amino acids, unsaturated fatty acids, adenosines. In turn, a large number of secondary intermediates with chemical activity from the light oxidation are formed, thus proteins, fats, nucleic acids and other important cellular components are destroyed, causing serious damage and dysfunction of many cells, eventually leading to tumor cell death due to irreversible damage. In addition, the photosensitizers also act on microvessels in tumor tissue, resulting in vascular endothelial damage and vascular blood stasis, thereby resulting in tumor tissue necrosis. The photosensitizers have the two kinds of effects, and have not only a direct killing effect on tumor cells, but also an effect of blocking tumor blood vessels to lead to the tumor tissue hypoxia and nutrient depletion, and prevent tumor cell proliferation and metastasis. In addition, the photosensitizer can produce an immune response, which can cause an increase of TNF, IL-IB and IL-2 in tissues, so that the local macrophage phagocytosis occurs. For example, a case of liver cancer patient was firstly treated by photodynamic therapy, a month later, the lesion part was resected. The resected specimens were subjected to microscopic examination. And, it was found that a large number of tumor cells became necrosis, while lobular structure of tumor-surrounding liver tissues was normal, liver cell cords arranged regularly. There were a large number of lymphocytes and part of eosinophils, macrophages between them. This showed that the photosensitizer caused an immune response. It can be seen that the active mechanism of photodynamic therapy is mainly to destroy the tumor blood vessels, induce tumor cell apoptosis and cause immune function, collectively and directly effect on the tumor.

Photodynamic therapy has certain selectivity to the target tissue, and it has good killing controllability, low toxic and side effect, and short treatment time, and can ensure the apoptosis of tumor cells under the condition as far as possible to reduce the damage of normal tissue, can protect the appearance and the function of important organs etc. Photodynamic therapy become another treatment besides traditional therapies i.e. surgery, radiotherapy and chemotherapy. Photodynamic therapy is applicable to many malignant tumors including esophageal cancer, lung cancer, brain tumors, head and neck cancer, eye tumor, pharyngeal cancer, chest wall tumor, breast cancer, pleural mesothelioma, liver cancer, gastric cancer, peritoneal sarcoma, bladder cancer, gynecological tumor, rectal cancer, skin cancer etc. Repeated treatment will not produce resistance. The early stage of primary tumor can be cured. For middle stage and advanced tumors, especially for patients who can not be subjected to surgery because of frail elderly, heart, lung and kidney insufficiency or hemophilia, photodynamic therapy can be used as a palliative treatment, relieve symptoms, reduce pain, improve the quality of life and prolong the survival time.

The photosensitizer is a key issue in the study of photodynamic therapy. The photosensitizers used in the existing PDT are basically porphyrin compounds such as Photofrin, the first anticancer photosensitizer developed by the Roswell Park Cancer Institute in U.S., its indications are superficial tumors suitable to be excited by 630 nm wavelength laser such as intracavitary tumors and skin cancer. The application of Photofrin in photodynamic therapy has nearly twenty years of history, has achieved very good therapeutic effects, and promoted the development of photodynamic therapy. But the Photofrin and its imitations such as Photosan and Photogem have significant shortcomings, they are composed of 8 or more porphyrin compounds, have no controllable quality standard, and have a large dark toxicity etc. Porphyrin compounds are characterized by conjugated double bonds in their structures, which make them to be an ideal light absorbing material, but at the same time make such compounds often hydrophobic. However, how to effectively and successfully transport drugs, especially hydrophobic drugs, to the tumor tissue area or the diseased area, has been challenging the creativity of scientists. Clinical practice has proved that the best is a water soluble compound, because water-solubility will speed up the transport of photosensitizer in vivo, shorten the time interval between the administration and the illumination. Commonly used method is to add surfactants, but the surfactants are likely to produce toxicity to the human body, so it is considered not to be perfect. In addition, the ideal photosensitizer should be amphipathic, and the two should achieve a better balance. The liposoluble property is favorable for it to penetrate cell membrane.

In photodynamic therapy, the photosensitizer is a key factor affecting the efficacy of photodynamic therapy. Therefore, the creation of new photosensitizers with high efficiency and low toxicity is of decisive significance to promote the development and clinical application of photodynamic therapy. Herein, the present invention provides a novel class of 2(4)-(1-hexyloxyethyl)-6,7-bis-propionate-1,3,5,8-tetramethyl-4 (2)-vinylporphyrin and its analogues.

### The Contents of Invention

In one aspect, the invention relates to dicarboxylate salt compounds of formulae:

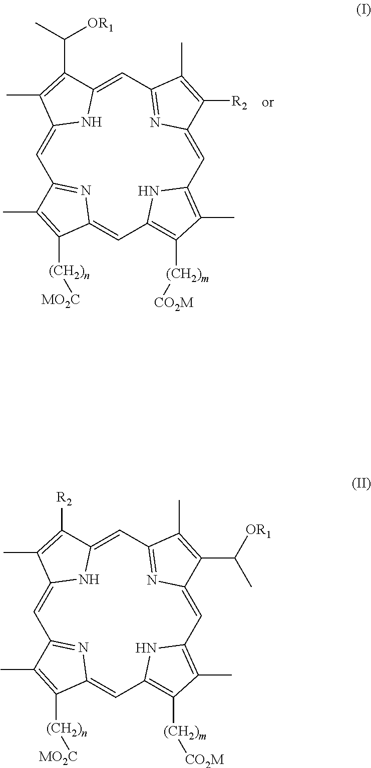

wherein
R₁ is selected from C₁₋₈ alkyl group;
R₂ is selected from the group consisting of C₁₋₈ alkyl group and C₂₋₈ alkenyl group;
M, at each occurrence, is independently selected from alkali metal and alkaline earth metal;
m is selected from an integer from 1 to 6;
n is selected from an integer from 1 to 6.

In one embodiment, the present invention relates to the dicarboxylate compounds of formula (I) or (II) as defined above, wherein R₁ is selected from pentyl, hexyl and heptyl, preferably n-hexyl.

In one embodiment, the present invention relates to the dicarboxylate compounds of formula (I) or (II) as defined above, wherein R₂ is selected from the group consisting of ethyl and vinyl groups.

In one embodiment, the present invention relates to the dicarboxylate compounds of formula (I) or (II) as defined above, wherein M, at each occurrence, is independently selected from the group consisting of alkali metal.

In one embodiment, the present invention relates to the dicarboxylate compounds of formula (I) or (II) as defined above, wherein M, at each occurrence, is independently selected from the group consisting of sodium or potassium.

In one embodiment, the present invention relates to the dicarboxylate compounds of formula (I) or (II) as defined above, wherein the two M are identical.

In one embodiment, the present invention relates to the dicarboxylate compounds of formula (I) or (II) as defined above, wherein m is 2.

In one embodiment, the present invention relates to the dicarboxylate compounds of formula (I) or (II) as defined above, wherein n is 2.

In one embodiment, the present invention relates to the dicarboxylate compounds of formula (I) or (II) as defined above, in which both m and n are 2.

In one embodiment, the present invention relates to dicarboxylate compounds of formula (I) or (II), wherein
R₁ is n-hexyl;
R₂ is vinyl;
M, at each occurrence, is independently selected from sodium;
m is 2;
n is 2.

In one embodiment, the present invention relates to a dicarboxylate compound of formula (I) or (II) compound having the following structure:

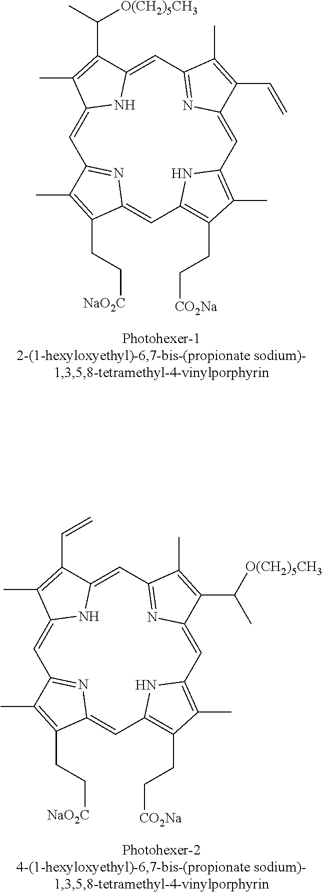

On the other hand, the present invention relates to a method for synthesizing photohexer-1 and/or photohexer-2, which comprises the following steps:
(i) treatment of protoporphyrin dimethyl ester with a base (e.g., alkali metal hydroxide, such as LiOH, NaOH, KOH; alkali metal carbonate, such as sodium carbonate, potassium carbonate) optionally in a solvent (preferably water) to provide 2(4)-(1-hydroxyethyl)-6,7-bis [2-(methoxycarbonyl) ethyl-1,3,5,8-tetramethyl-4(2)-vinylporphyrin (i.e. HVD-1 and HVD-2),
(ii) optional separation of HVD-1 and HVD-2;
(iii) reaction of HVD-1 and/or HVD-2 with n-hexanol in the presence of hydrogen halide (e.g. HCl, HBr, HI) in a solvent (preferably organic solvents, such as dichloromethane, chloroform, tetrahydrofuran, diethyl ether, benzene) to provide the product 2-(1-hexyloxyethyl)-6,7-bis [2-(methoxycarbonyl)ethyl]-1,3,5,8-tetramethyl-4-vinylporphyrin and/or 4-(1-hexyloxyethyl)-6,7-bis [2-(methoxycarbonyl)ethyl]-1,3,5,8-tetramethyl-2-vinyl porphyrin;
(iv) then treatment of the product obtained in step (iii) with a base (e.g., alkali metal hydroxide, such as LiOH, NaOH, KOH, preferably sodium hydroxide; alkali metal carbonate, such as sodium carbonate, potassium carbonate) in a solvent (such as an alcohol, such as ethanol, isopropanol; water) to provide photohexer-1 and photohexer-2 as sodium salts.

Compound(s) of formula (I) or (II) as defined above (also known as “the compound(s) of the present invention”) are a class of new photosensitizers for photodynamic therapy, such as for the treatment or diagnosis of malignant tumors, precancerous lesions or benign lesions, have significant advantages as the photosensitizer, and have the following conditions that ideal photosensitizers should have: first, the compound of the present invention is a single compound, its quality is controllable; second, the compound of the invention has strong targeting property to tumor tissue; third, the compound of the present invention has strong photodynamic activity, high anticancer activity, not only has direct killing effect on tumor cells, but also has the effect of blocking tumor blood vessels; fourth, the compound of the present invention absorbs light at longer wavelength, so it has strong penetrating power to the tumor tissue, thereby can treat not only superficial tumors, but also deep tumors; fifth, the retention time of the compound of the invention in the skin is short and the dark toxicity is small; sixth, the compound of the present invention is soluble in water and is easy to be made into formulation without additional complex preparation processes; seven, the compound of the present invention is stable in nature.

The inventors devoted to the study of looking for the ideal photosensitizer, and surprisingly found that the preparation of carboxylic groups or methyl ester thereof in structure of porphyrins into the corresponding carboxylate salt such as sodium carboxylate produced a great influence on the solubility of the compounds, even the activity thereof. If there are more than two carboxylate salt groups in the structure, the compound has water-soluble property, and the activity is significantly improved. If there is only one carboxylate salt group in the structure, the compound can only be slightly soluble in water, and can not reach the requirement of preparing water soluble preparation. In addition, when transmission of such photosensitizers to the tumor tissue region or the lesion region, since the region is lactic acid environment, the photosensitizer will deposit therein. This is one of the reasons why the photosensitizer targets the tumor tissue. While, carboxylate salt groups in part of the photosensitizer will be hydrolyzed into carboxylic acids in the acidic environment. That is, it will become hydrophobic compounds, which is favourable for penetrating the membrane of tumor cells. Therefore, the compound of the present invention is a photosensitizer with potential value in the treatment of malignant tumor and benign lesions.

Due to the excellent physical and chemical properties of the compounds of the invention, as shown in the examples below, they can be directly prepared into lyophilization powder preparations to facilitate production, storage, transportation and use without addition of any excipient.

In another aspect, the invention relates to a compound of the invention useful as a medicament, especially as a photosensitizer.

In another aspect, the present invention relates to a pharmaceutical composition comprising the compounds of the invention, the composition is used as a photosensitizer, for example as a photosensitizer for the treatment or diagnosis of malignant tumors, precancerous lesions and benign lesions.

In another aspect, the present invention relates to use of compounds of the invention in the manufacture of a medicine for use as a photosensitizer, for example as a photosensitizer for the treatment or diagnosis of malignant tumors, precancerous lesions and benign lesions.

In another aspect, the invention relates to a method for the treatment or diagnosis of malignant tumors, precancerous lesions and benign lesions, which includes administering one or more compounds of the present invention to a subject in photodynamic therapy.

### Definition

The “alkali metal” mentioned in the description and the claims refers to metal elements from the Group IA of the elements periodic table, including lithium, sodium, potassium, rubidium and cesium, preferably sodium and potassium, more preferably sodium.

The alkaline earth metal described in the description and the claims refers to metal elements from the Group HA of the elements periodic table, including magnesium, calcium, strontium, barium, wherein calcium and magnesium are preferred.

The term “alkyl” used herein refers to the linear or branched alkyl groups. “C₁₋₈ alkyl” refers to a linear or branched alkyl group with 1-8 carbon atoms. The term includes, but is not limited to, the following groups: methyl, ethyl, propyl, butyl, pentyl, hexyl, heptyl, etc.

The term “alkenyl” used herein refers to a linear or branched alkyl group having at least one double bond (as appropriate, presenting E or Z stereochemistry). “C₂₋₈ alkenyl” refers to linear or branched alkenyl groups having 2-8 carbon atoms, including but not limited to vinyl, propenyl, 2-propenyl, 1-butenyl, 2-butenyl, 3-buteny, 1-pentenyl, 2-pentenyl, 3-pentenyl, 1-hexenyl, 2-hexenyl and 3-hexenyl.

The “malignant tumor” mentioned in the description and the claims refers to malignant solid tumors, including but not limited to oral and maxillofacial cancer, nasopharyngeal carcinoma, esophageal cancer, gastric cancer, bile duct cancer, colon cancer, rectal cancer, skin cancer, lung cancer, bronchial carcinoma, breast cancer and subcutaneously metastatic nodules after resection of breast cancer, cervical cancer, liver cancer, bladder cancer, pleural mesothelioma, pancreatic cancer, cancer of the penis, perianal tumor and residual cancer after resection of perianal tumor, Kaposi's sarcoma, prostate cancer, melanoma and brain tumors.

The “precancerous lesions” mentioned in the description and the claims refers to the lesions with cancerous tendency, such as Barrett's esophagus, oral leukoplakia, etc.

The “benign lesions” mentioned in the description and claims refers to the lesions without cancerous tendency, such as nevus flammeus, age-related macular degeneration, atherosclerosis, rheumatoid arthritis, skin microvascular malformation, psoriasis, lupus erythematosus skin lessions etc.

The term “treat” or “treatment” used in this description and the claims refers to a preventive or therapeutic treatment, including the prevention, mitigation, delay, and cure of indications or symptoms thereof.

The term “diagnosis” used in this description and the claims refers to identification of a disease or its type.

Within the description and the claims, the term “subject” refers to mammals, including but not limited to pets such as cats, dogs etc.; farm animals, such as cattle, horses, sheep, ect.; primates, such as chimpanzees, human etc.; and human is preferred.

### Dosage and Administration

The compound of the invention can be formulated into any suitable dosage form and can be administered in any suitable way. The compounds of the invention can be prepared as solution, suspension, emulsion, lyophilized preparation and the like for injection (e.g., intraarterial, intravenous, intramuscular, subcutaneous, intraperitoneal injection etc.) or infusion application; as tablets, solution, capsulesfor oral administration; as ointment, cream, suppository, patches etc. for topical application to the skin or mucosa; as aerosol, spray and powder for inhalation application. The preferred methods of administration are generally injection/infusion administration and topical application of skin or mucous membranes.

The methods and the auxiliary materials for preparing the compound of the invention into a dosage form are routine methods and auxiliary materials well known by a skilled person in the art. For example, for injection, appropriate auxiliary materials are such as water, sodium chloride, glucose, glycerol, pH buffer and the like. More detailed information about the dosage forms and auxiliary materials can be found in the reference books in the art, such as Luo Mingsheng, Gao Tianhui, editor in chief, Yaoji Fuliao Daquan (Comprehensive Pharmaceutical Auxiliary Materials), the second edition, Sichuan science and technology press. The skilled person in the pharmaceutical field can adjust the formulations within the teachings of the present specification to provide a variety of formulations for a particular route of administration without destabilizing the compounds of the invention or damaging their therapeutic activity.

In general, for a mammal such as a human, an effective amount of a compound of the invention is 0.001-100 mg/kg body weight, preferably 0.005-20 mg/kg body weight, more preferably 0.01-5 mg/kg body weight, even more preferably 0.05-2 mg/kg body weight. However, it should be understood that the effective amount of the compound of the present invention will be determined by the researchers or clinicians according to a reasonable medical judgment. A specific effective amount will depend on many factors, for example, type and severity of the disease to be treated; the specific compounds used; the fluence and irradiation time; the age, body weight and general health status of the subject; duration of treatment; administration in combination; and other factors well known in the field of medicine. In some cases, the effective amount may be higher than the upper limit or lower than the lower limit of the above range.

The compounds of the present invention can be used in combination with any matchable excitation light sources knowin in the art. For the compound of the present invention, the irradiation wavelength is preferably 627±10 nm (i.e., irradiation wavelength can vary within the range of 617-637 nm), more preferably 627±3 nm (i.e., irradiation wavelength can vary in the range of 624-630 nm). The dose of light is preferably 50-300 Joules/cm².

### DESCRIPTION OF FIGURES

FIG. 1: HPLC chromatogram of protoporphyrin dimethylester.

FIG. 2: HPLC chromatogram of 2(4)-(1-hydroxyethyl)-6,7-bis [2-(methoxycarbonyl)ethyl]-1,3,5,8-tetramethyl-4(2)-vinylporphyrin (HVD-1 and HVD-2).

FIG. 3: HPLC chromatogram of 2-(1-hydroxyethyl)-6,7-bis [2-(methoxycarbonyl)ethyl]-1,3,5,8-tetramethyl-4-vinylporphyrin (HVD-1).

FIG. 4: HPLC chromatogram of 4-(1-hydroxyethyl)-6,7-bis[2-(methoxycarbonyl)ethyl]-1,3,5,8-tetramethyl-2-vinylporphyrin (HVD-2).

FIG. 5: HPLC chromatogram of 2-(1-hexyloxyethyl)-6,7-bis[2-(methoxycarbonyl)ethyl]-1,3,5,8-tetramethyl-4-vinylporphyrin.

FIG. 6: HPLC chromatogram of 2-(1-hexyloxyethyl)-6,7-di(propionate sodium)-1,3,5,8-tetramethyl-4-vinylporphyrin.

FIG. 7: HPLC chromatogram of 4-(1-hexyloxyethyl)-6,7-bis[2-(methoxycarbonyl)ethyl]-1,3,5,8-tetramethyl-2-vinylporphyrin.

FIG. 8: HPLC chromatogram of 4-(1-hexyloxyethyl)-6,7-di(propionate sodium)-1,3,5,8-tetramethyl-2-vinylporphyrin.

FIG. 9: The killing effect of photohexer-1 (P-1) mediated photodynamic therapy on SGC7901 cells.

FIG. 10: The killing effect of photohexer-2 (P-2) mediated photodynamic therapy on SGC7901 cells.

FIG. 11: The killing effect of photohexer-1 (P-1) mediated photodynamic therapy on SW-480 cells.

FIG. 12: The killing effect of photohexer-2 (P-2) mediated photodynamic therapy on SW-480 cells.

FIG. 13: The killing effect of photohexer-1 (P-1) mediated photodynamic therapy on SW-620 cells.

FIG. 14: The killing effect of photohexer-2 (P-2) mediated photodynamic therapy on SW-620 cells.

FIG. 15: The killing effect of photohexer-1 (P-1) mediated photodynamic therapy on Caco2 cells.

FIG. 16: The killing effect of photohexer-2 (P-2) mediated photodynamic therapy on Caco2 cells.

FIG. 17: The killing effect of photohexer-1 (P-1) mediated photodynamic therapy on Eca-109 cells.

FIG. 18: The killing effect of photohexer-2 (P-2) mediated photodynamic therapy on Eca-109 cells.

FIG. 19: The killing effect of photohexer-1 (P-1) mediated photodynamic therapy on MDA-MB-231 cells.

FIG. 20: The killing effect of photohexer-2 (P-2) mediated photodynamic therapy on MDA-MB-231 cells.

FIG. 21: The killing effect of photohexer-1 (P-1) mediated photodynamic therapy on MCF-7 cells.

FIG. 22: The killing effect of photohexer-2 (P-2) mediated photodynamic therapy on MCF-7 cells.

FIG. 23: The killing effect of photohexer-1 (P-1) mediated photodynamic therapy on MCF-7/ADR cells.

FIG. 24: The killing effect of photohexer-2 (P-2) mediated photodynamic therapy on MCF-7/ADR cells.

FIG. 25: The killing effect of photohexer-1 (P-1) mediated photodynamic therapy on CT26 cells.

FIG. 26: The killing effect of photohexer-2 (P-2) mediated photodynamic therapy on CT26 cells.

FIG. 27: The killing effect of photohexer-1 (P-1) mediated photodynamic therapy on 7721 cells.

FIG. 28: The killing effect of photohexer-2 (P-2) mediated photodynamic therapy on 7721 cells.

FIG. 29: Fluorescence microscopy of P-1, P-2 in cells.

FIG. 30A: The graph of tumor volume after the treatment of P1, P2 combined with light irradiation.

FIG. 30B: The tumor volume after the treatment of P1 combined with light irradiation.

FIG. 30C: The tumor volume after the treatment of P2 combined with light irradiation.

FIG. 31A: The graph of body weight of mice after the treatment of P-1, P-2, combined with light irradiation.

FIG. 31B: The graph of body weight of mice after the treatment of P-1 combined with light irradiation.

FIG. 31C: The graph of body weight of mice after the treatment of P-2 combined with light irradiation.

FIG. 32: Visual picture of 4T1 tumor-bearing mice after the treatment of P-1, P-2 combined with light irradiation.

FIG. 33A: Visual picture of heart of 4T1 tumor-bearing mice after the treatment of P-1, P-2 combined with light irradiation (including weight).

FIG. 33B: Visual picture of liver of 4T1 tumor-bearing mice after the treatment of P-1, P-2 combined with light irradiation (including weight).

FIG. 33C: Visual picture of spleen of 4T1 tumor-bearing mice after the treatment of P-1, P-2 combined with light irradiation (including weight).

Compared with the blank control mice, there were no obvious changes in the other organs except the spleen had obvious enlargement.

FIG. 33D: Visual picture of lung of 4T1 tumor-bearing mice after the treatment of P-1, P-2 combined with light irradiation (including pulmonary nodules and weight).

FIG. 33E: Visual picture of kidney of 4T1 tumor-bearing mice after the treatment of P-1, P-2 combined with light irradiation (including weight).

FIG. 33F: Visual picture of tumor of 4T1 tumor-bearing mice after the treatment of P-1, P-2 combined with light irradiation (including tumor inhibition rate).

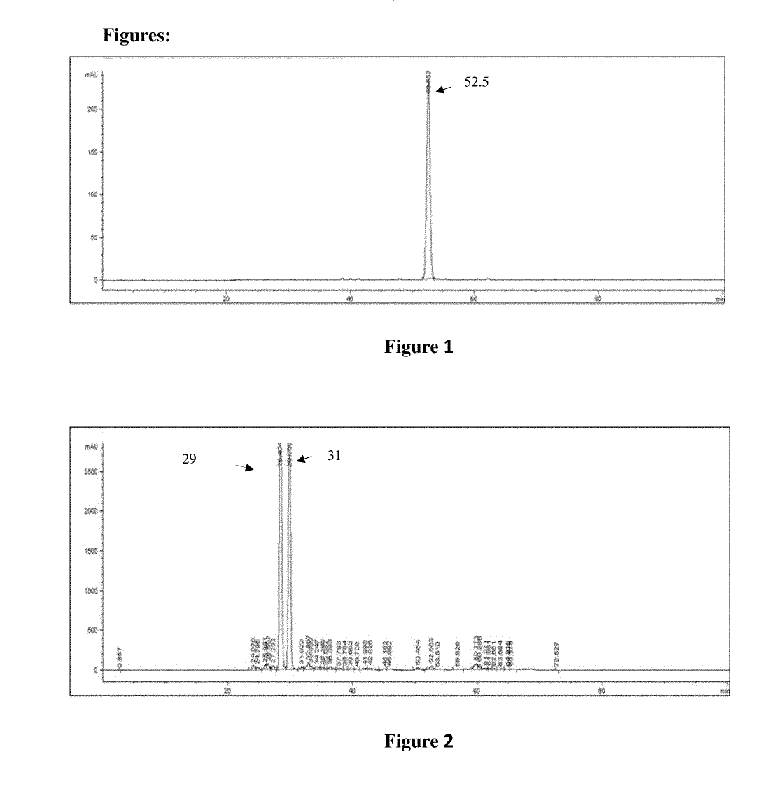

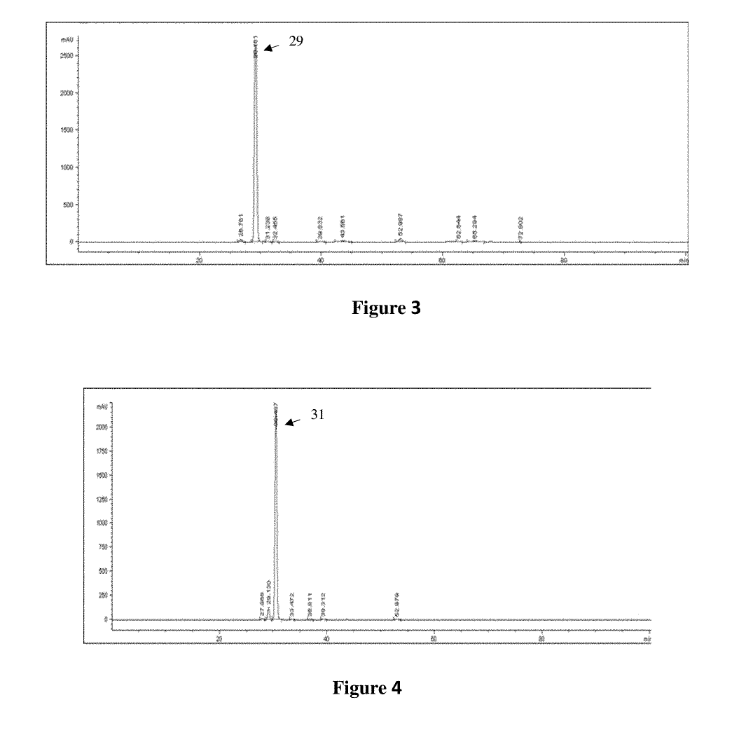

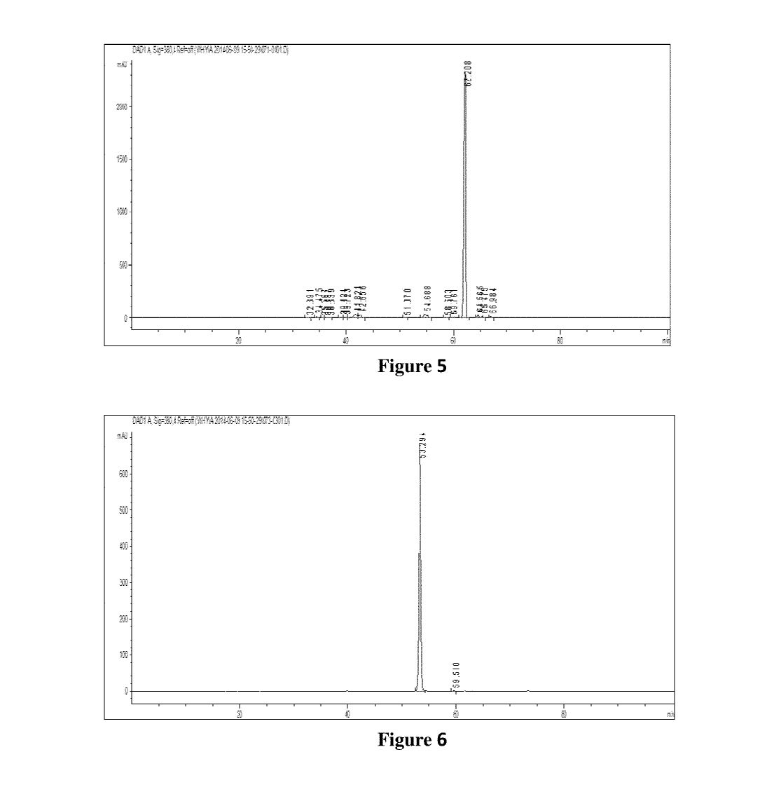

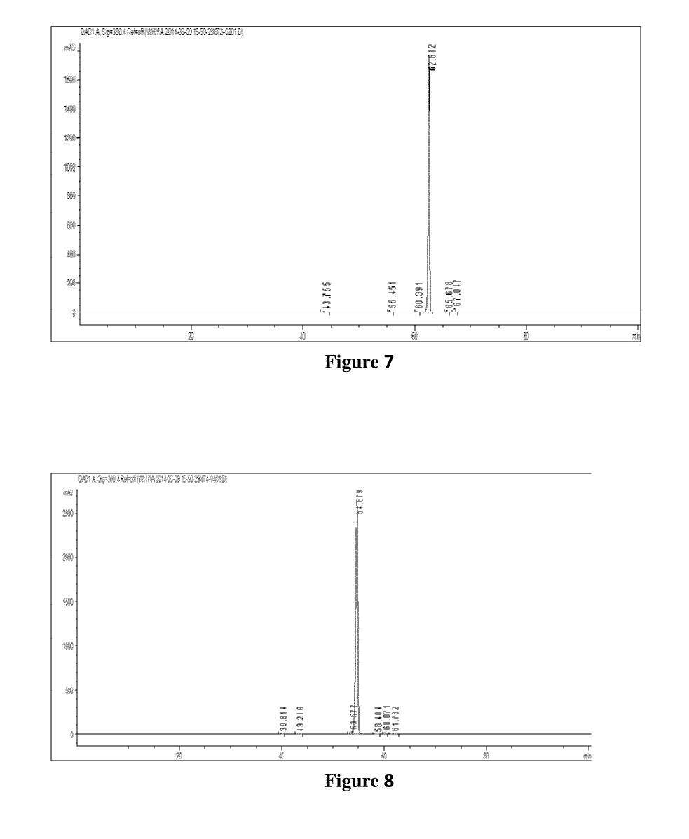

### EXAMPLES

The following examples are used to elaborate the present invention in greater detail, but they should not be misinterpreted as limitations of the scope defined by the claims of the present invention.

### Experimental Instruments and HPLC Condition

1 Experimental Instruments:

UV absorption spectrum was determined on Heλiosα type UV spectrophotometer. THERMO SPECTRONIC Co.). Infrared spectrum was recored on Nicolet 5700 Fourier transform infrared spectrometer (KBr tablet) THERMO Co.). The NMR spectra were measured with the AVIIIHD type 600 nuclear magnetic resonance spectrometer (TMS as internal standard) (Bruck Co.). High resolution mass spectrometry and cold spray MS were obtained on Acuu TOF CS type mass spectrometer (JMS-T100CS, JEOL, Japan).

HPLC analysis was performed with Agillent 1200 Series HPLC analyzer:

2HPLC Condition

Analysis condition: analysis column: Japan Shiseido Capcell C₁₈ MG 4.6 mm×150×5 μm; detection wavelength: 380 nm; column temperature: 30° C.; sample: the sample was dissolved with methanol, and filtered using 0.45 m nylon microporous membrane before injection; mobile phase: methanol and 1% acetic acid in water; flow rate: 1 mL/min.

| Time (min) | A: 1% acetic acid | B: methanol (%) | in water (%) |
|---|---|---|---|
| 0.0 | 30.0 | 70.0 | |
| 30.0 | 10.0 | 90.0 | |
| 45.0 | 10.0 | 90.0 | |
| 60.0 | 0.0 | 100.0 | |
| 70.0 | 0.0 | 100.0 | |
| 70.5 | 30.0 | 70.0 | |
| 100.5 | 30.0 | 70.0 | |

(The last 30 minutes is for the equilibrium of column, and ready for the next sample)

Preparative separation conditions: column chromatography with silica gel H (fineness 200-300 mesh, Qingdao marine chemical plant) is used for the preparative separation of reaction products.

### Preparation of Intermediates

### Preparation 1. Preparation of 2(4)-(1-hydroxyethyl)-6,7-bis [2-(methoxycarbonyl)ethyl]-1,3,5,8-tetramethyl-4(2)-vinylporphyrin (HVD-1 and HVD-2)

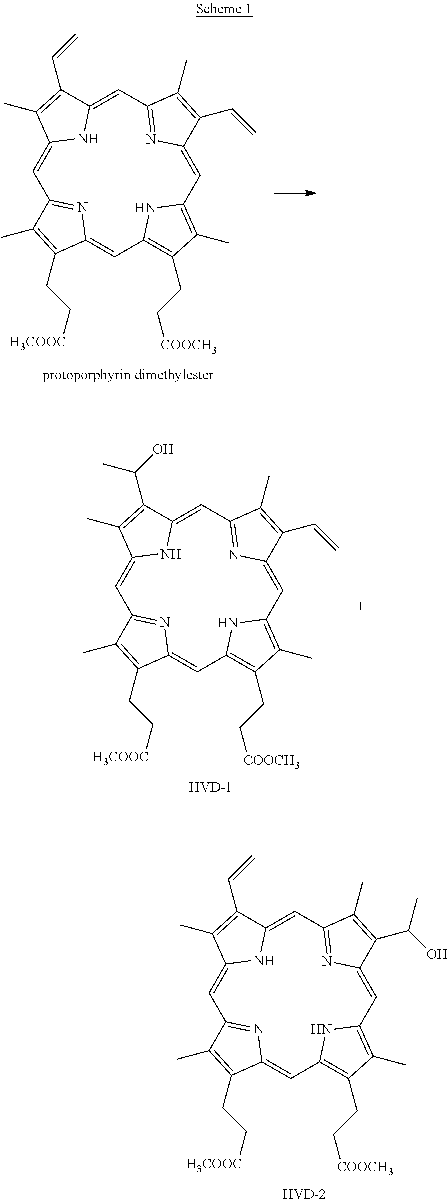

To 500 ml hydrochloric acid, protoporphyrin dimethylester 100 g was added, stirring to dissolve. The solution was stirred in 25° C. water bath for 6 hours. After that, 20% sodium hydroxide solution (2800 ml) was added at a constant rate and the pH value of the solution should be 13. The solution was allowed to stand for 1 hour. Then 500 ml acetic acid was added until pH was 4-5. After reaction for more 30 min and filtration by suction, the residue was washed with water, dried by suction, and further dried in a dryer. 95 g protoporphyrin dimethylester derivatives were obtained. To protoporphyrin dimethylester derivatives (95 g) a solution of 5% sulfuric acid in methanol (1000 ml) was added, stirred at room temperature for 30 minutes. The reaction solution was neutralized to pH 7 with (NH₄)₂CO₃. Then the solution was concentrated under reduced pressure. The concentrated residue was extracted with dichloromethane. The extracted solution was washed with appropriate amount of water and dehydrated by anhydrous sodium sulfate, filtered. Then, dichloromethane was recovered under vacuum to provide the protoporphyrin dimethylester derivatives.rotoporphyrin dimethylester derivatives were dissolved in appropriate amount of acetone. Silica gel 300 g was added and homogenously stirred. Then, acetone was evaporated. The resulting dry silicon gel powder was homogenously added to the top of the prefilled silicon gel chromatographic column (1600 g silicon gel, 200-300 mesh, pre-equilibrated by 0.2% methanol in dichloromethane). Then, 0.2% methanol in dichloromethane was added to separate. Fractions were collected at 200 ml per fraction, at the same time, the total percentage content of 2(4)-(1-hydroxyethyl)-6,7-bis[2-(methoxycarbonyl)ethyl]-1,3,5,8-tetramethyl-4(2)-vinylporphyrin in each fraction was detected by HPLC. For the fractions with less than 90% of purity, after removing solvent under reduced pressure, they were refined with isopropanol and acetone.

### Preparation 2. The separation and purification of 2-(1-hydroxyethyl)-6,7-bis[2-(methoxycarbonyl)ethyl]-1,3,5,8-tetramethyl-4-vinylporphyrin} (HVD-1) and 4-(1-hydroxyethyl)-6,7-bis[2-(methoxycarbonyl) ethyl]-1,3,5,8-tetra-methyl-2-vinylporphyrin} (HVD-2)

1000 g of silicon gel (Fineness 200-300 mesh) was weighed, to which methylene chloride was added and homogenously stirred. The mixture was filled into glass chromatographic column with 60 mm diameter. Separately, 50 g of 2(4)-(1-hydroxyethyl)-4(2)-vinylpyroporphyrin dimethyl ester was weighed, and an appropriate amount of methylene chloride was added to dissolve. Loading the sample by a wet process, using methylene chloride as mobile phase, fractions were collected and monitored by HPLC, to provide respectively:

2-(1-hydroxyethyl)-6,7-bis[2-(methoxycarbonyl)ethyl]-1,3,5,8-tetramethyl-4-vinylporphyrin (HVD-1): ¹H NMR (CDC₁₃) δ10.23, 10.09 (each s, 1H, 2 meso H); 9.89 (s, 2H, 2 meso H); 8.27 (m, 1H, CH═CH₂); 6.35, 6.19 (each d, 1H, CH═CH₂); 6.18 (q, 1H, CH (OH) (CH₃); 4.30 (m, 4H, 2CH₂CH₂CO₂CH₃); 3.67 (s, 6H, 2CO₂CH₃); 3.58, 3.49 (each s, 3H, 2CH₃); 3.51 (s, 6H, 2CH₃); 3.22 (t, 4H, 2CH₂CH₂CO₂CH₃); 2.08 (d, 3H, CH (OH) CH₃). HR-ESI-MS m/z: C₃₆H₄₀N₄O₅ calculated value: 609.3136 (M+1), measured value: 609.3163 (M+1). These data were in agreement with those reported in literature.

4-(1-hydroxyethyl)-6,7-bis[2-(methoxycarbonyl)ethyl]-1,3,5,8-tetramethyl-2-vinylporphyrin (HVD-2): ¹H NMR (CDCl₃) δ10.21, 10.04, 9.96, 9.94 (each s, 1H, meso, H); 8.21 (m, 1H, CH═CH₂); 6.36, 6.18 (each d, 1H, CH═CH₂); 6.23 (q, 1H, CH (OH) CH₃); 4.36 (m, 2H, 2CH₂CH₂CO₂CH₃); 3.68 (s, 6H, 2CO₂CH₃ and 1CH₃); 3.56, 3.53, 3.45 (each s, 3H, 3CH₃); 3.25 (t, 4H, 2CH₂CH₂CO₂CH₃); 2.10 (d, 3H, CH (OH) CH₃). HR-ESI-MS). m/z: C₃₆H₄₀N₄O₅ calculated value: 609.3136 (M+1), measured value: 609.3112 (M+1). These data were in agreement with those reported in literature.

HPLC retention time of HVD-1: 29 min, purity: 94%; HPLC retention time of HVD-2: 31 min, purity: 96%.

### Example 1: Preparation of 2-(1-hexyloxyethyl)-6,7-bis(propionate sodium)-1,3,5,8-tetramethyl-4-vinylporphyrin (Photohexer-1) i.e. 2-(1-hexyloxyethyl)-6,7-bis[2-(sodium carbonate)ethyl]-1,3,5,8-tetramethyl-4-vinylporphyrin (Photohexer-1)

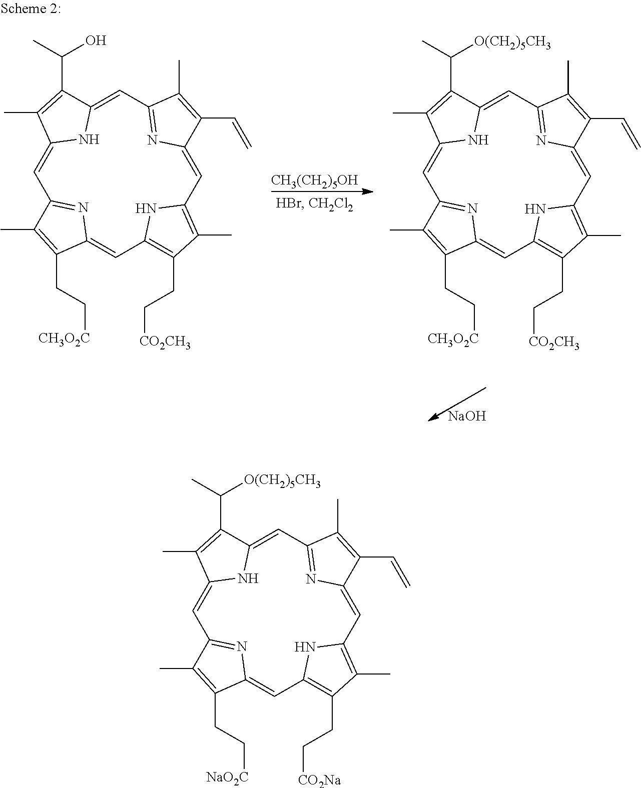

### Step 1

### Synthesis of 2-(1-hexyloxyethyl)-6,7-bis[2-(methoxycarbonyl)ethyl]-1,3,5,8-tetramethyl-4-vinylporphyrin

Take 2-(1-hydroxyethyl)-6,7-bis[2-(methoxycarbonyl)ethyl]-1,3,5,8-tetramethyl-4-vinylporphyrin (HVD-1, 100 mg), dissolve it in 8 mL of anhydrous dichloromethane. Then to this solution 1.5 ml 1-hexanol was added, subsequently 2.5 ml dichloromethane solution saturated with gaseous hydrogen bromide was added, gently mixed well, plugged tightly with a glass stopper, allowed to stand in the dark at room temperature for 1 hours, when necessary, the reaction time may be prolonged. After that, 20 ml water/dichloromethane (1:1) solution was added and the organic layer was separated, washed with water. After removing water by adding an approprioate amount of anhydrous sodium sulfate, the organic solvent was evaporated under reduced pressure. Then the crude product was separated by silica gel column chromatography, firstly using 0.25% methanol in dichloromethane to elute protoporphyrin dimethyl ester, then using 0.75% methanol in dichloromethane to seperate, to afford dark red powder. Retention time: 62.2 min., purity: 88.9% (by HPLC detection).

### Spectral Analysis Detection:

¹H NMR (CDC₁₃): 5:10.65, 10.19, 10.09 D, 10.04 (each s, 1H, 4 meso H) 8.30 (m, 1H, CH═CH₂); 6.37, 6.18 (each d, 1H, CH═CH₂); 6.11 (q, 1H, CH₃CHOCH₂C₅H₁₁); 4.40 (m, 4H, CH₂CH₂CO₂CH₃); 3.73, 3.69 (diester CH₃); 3.65, 3.63) (each d, 6H, 4×CH₃); 3.28 (t, 4; H, CH₂CH₂CO₂CH₃) 2.26 (d, 3H, CH₃CHOCH₂C₅H₁₁); 0.71-1.79 (m, 11H, CH₃CHOCH₂C₅H₁₁); −3.67 (s, 2H, NH).

HR-ESI-MS m/z: C₄₂H₅₃N₄O₅ calculated value: 693.4010 (M+1), measured value: 693.4010 (M+1).

The above measured data were in agreement with the chemical structure of 2-(1-hexyloxyethyl)-6,7-bis[2-(methoxycarbonyl)ethyl]-1,3,5,8-tetra-methyl-4-vinyl-porphyrin.

### Step 2: The preparation of 2-(1-hexyloxyethyl)-6,7-bis(propionate sodium)-1,3,5,8-tetramethyl-4-vinylporphyrin (Photohexer-1) i.e. {2-(1-hexyloxyethyl)-6,7-bis[2-(sodium carbonate)ethyl]-1,3,5,8-tetramethyl-4-vinylporphyrin} (Photohexer-1)

70 mg 2-(1-hexyloxyethyl)-6,7-bis[2-(methoxycarbonyl)ethyl]-1,3,5,8-tetra-methyl-4-vinylporphyrin was dissolved in 42 ml diethyl ether solution. Under stirring, a solution of sodium hydroxide in isopropanol (50 mg NaOH was dissolved in 15 ml iso-PrOH) was added dropwise. A capillary point sample tube was used to absorb the reaction solution, and the color detection reaction around the precipitate was observed on the filter paper. When the solution around the precipitate on the filter paper is colorless, stop adding. The reaction solution was transferred to a centrifuge tube, and centrifuged for 10 minutes (2500 RPM) in a centrifuge. The supernatant was decanted and the solid in the centrifuge tube was placed in the vacuum dryer for drying, to provide dark red powder. HPLC detection, retention time: 53.3 minutes, purity: 99.6%.

### Spectral Analysis Detection:

UV-vis [CH₃OH, λmax (nm)]: 624, 571, 535, 499, 398, 204;

IR(KBr): 3398, 3309, 2953, 2927, 2857, 1555, 1414, 1100, 909, 735, 678 cm⁻¹;

¹H NMR (CDC₁₃) δ: 10.65, 10.26, 10.20, 10.17 (each s, 1H, 4 meso H) 8.33 (m, 1; H, CH═CH₂;

6.16, 6.14 (each d, 1H, CH═CH₂); 6.14 (q, 1; H, CH₃CHOCH₂C₅H₁₁) 4.39 (m, 4H. CH₂CH₂CO₂CH₃); 3.76 (q, 2H, CH₃CHOCH₂C₅H₁₁); 3.74, 3.73, 3.66, 3.64 (each s, 3H, 4×CH₃); 3.08 (t, 4; H, CH₂CH₂CO₂CH₃) 2.21 (d, 3H, CH₃CHOCH₂C₅H₁₁); 0.58-1.75 (m, 11H, CH₃CHOCH₂C₅H₁₁).

CS-MS m/s: 709.39 [M+1]⁺, 687.39 [M+2H-Na]⁺. HR-ESI-MS m/z: 709.3356 gives the excimer ion peak [M+H]⁺ corresponding to the molecular formula C₄₀H₄₇N₄O₅Na₂ (calculated value 709.3342), degree of unsaturation is 18.5.

According to the analytical results of the above spectral data, the final product was in agreement with the chemical structure of 2-(1-hexyloxyethyl)-6,7-bis(propionate sodium)-1,3,5,8-tetramethyl-4-vinyl porphyrin.

### Example 2

### The preparation of 4-(1-hexyloxyethyl)-6,7-bis(propionate sodium)-1,3,5,8-tetramethyl-2-vinylporphyrin (Photohexer-2) i.e. {4-(1-hexyloxyethyl)-6,7-bis[2-(sodium carbonate)ethyl]-1,3,5,8-tetramethyl-2-vinylporphyrin} (Photohexer-2)

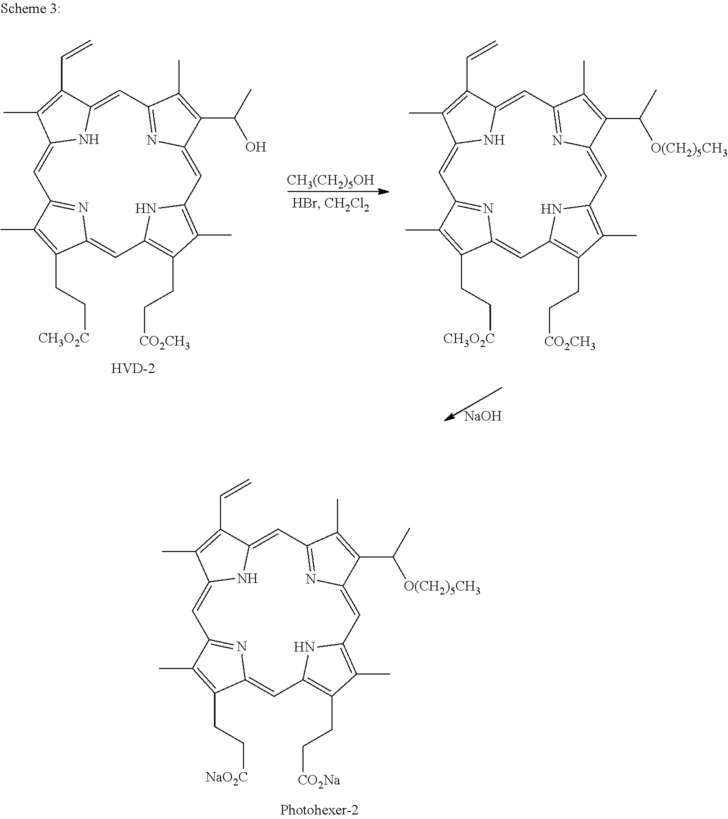

### Step 1

### Synthesis of 4-(1-hexyloxyethyl)-6,7-bis[2-(methoxycarbonyl)ethyl]-1,3,5,8-tetramethyl-2-vinylporphyrin

Take 4-(1-hexyloxyethyl)-6,7-bis[2-(methoxycarbonyl)ethyl]-1,3,5,8-tetramethyl-2-vinylporphyrin (HVD-2, 100 mg), dissolve it in 8 mL of anhydrous dichloromethane. To this solution 1.5 ml of 1-hexanol was added, subsequently 2.5 ml dichloromethane solution saturated with gaseous hydrogen bromide was added, gently mixed well, plugged tightly with a glass stopper, allowed to stand in dark at room temperature for 1 hours, when necessary, the reaction time may be prolonged. After that, 20 ml water/dichloromethane (1:1) solution was added and the organic layer was separated, washed with water. After removing water by adding an appropriate amount of anhydrous sodium sulfate, the organic solvent was evaporated under reduced pressure. Then the crude product was separated by silica gel column chromatography, firstly using 0.25% methanol in dichloromethane to elute protoporphyrin dimethyl ester, then using 0.75-1% methanol in dichloromethane to separate, to afford dark red powder, retention time: 62.6 min., purity: 91.8% (by HPLC detection).

### Spectral Analysis Detection:

¹H NMR (CDC₁₃): 10.61, 10.25, 10.09, 10.07 (each s, 1H, 4 meso H) 8.29 (m, 1H, CH═CH₂); 6.37 6.18 (each d, 1H, CH═CH₂); 6.13 (q, 1H, CH₃CHOCH₂C₅H₁₁) 4.43 (m, 4H. CH₂CH₂CO₂CH₃); 3.74, 3.69; (diester CH₃) 3.65 (q, 2H, CH₃CHOCH₂C₅H₁₁); 3.67, 3.66, 3.65, 3.63 (each s, 3H, 4×CH₃); 3.30 (t, 4H, CH₂CH₂CO₂CH₃) 2.27 (d, 3; H, CH₃CHOCH₂C₅H₁₁; 0.73-1.80 (m, 11H, CH₃CHOCH₂C₅H₁₁); −3.67 (s, 2H, 2 NH);

HR-ESI-MS m/z: C₄₂H₅₃N₄O₅ calculated value: 693.4010 (M+1), measured value: 693.4046 (M+1).

The above measured data were in agreement with the chemical structure of 4-(1-hexyloxyethyl)-6,7-bis[2-(methoxycarbonyl)ethyl]-1,3,5,8-tetramethyl-2-vinylporphyrin.

### Step 2

### The preparation of 4-(1-hexyloxyethyl)-6,7-bis(propionate sodium)-1,3,5,8-tetramethyl-2-vinylporphyrin (Photohexer-2) i.e. {4-(1-hexyloxyethyl)-6,7-bis[2-(sodium carbonate)ethyl]-1,3,5,8-tetramethyl-2-vinylporphyrin} (Photohexer-2)

70 mg 4-(1-hexyloxyethyl)-6,7-bis[2-(methoxycarbonyl)ethyl]-1,3,5,8-tetramethyl-2-vinylporphyrin was dissolved in 42 ml diethyl ether solution. Under stirring, a solution of sodium hydroxide in isopropanol (50 mg NaOH was dissolved in 15 ml iso-PrOH) was added dropwise. A capillary point sample tube was used to absorb the reaction solution, and the color detection reaction around the precipitate was observed on the filter paper. When the solution around the precipitate on the filter paper is colorless, stop adding. The reaction solution was transferred to a centrifuge tube, and centrifuged for 10 minutes (2500 RPM) in a centrifuge. The supernatant was decanted and the solid in the centrifuge tube was placed in the vacuum dryer for drying, to provide dark red powder. HPLC detection, retention time: 54.6 minutes, purity: 97.3%.

### Spectral Analysis Detection:

UV-vis [CH₃OH, λmax (nm)]: 624, 571, 535, 499, 398, 286;

IR(KBr): 3391.6, 3310.4, 2927.7, 2857.5, 1729.1, 1561.0, 1447.4, 1425.0, 1102.2, 838.5, 726.2, 702.8, 678.8 cm⁻¹;

¹H NMR (CDCl₃): 10.58, 10.25, 9.92, 9.63 (each s, 1H, 4 meso H) 7.91 (m, 1; H, CH═CH2); 6.06, 6.03 (each d, 1H, CH═CH₂); 6.06 (q, 1H, CH₃CHOCH₂C₅H₁₁) 4.40 (m, 4H. CH₂CH₂CO₂CH₃; 3.75 (q, 2H, CH₃CHOCH₂C₅H₁₁); 3.64, 3.61, 3.58, 3.48 (each s, 3H, 4×CH₃); 3.10 (t, 4; H, CH₂CH₂CO₂CH₃) 2.20 (d, 3H, CH₃CHOCH₂C₅H₁₁); 0.61-1.75 (m, 11H, CH₃CHOCH₂C₅H₁₁).

CS-MS m/s: 709.39 [M+1]+, 687.39 [M+2H-Na]⁺. HR-ESI-MS m/z: 709.3551 gives the excimer ion peak [M+H]⁺ corresponding to the molecular formula C₄₀H₄₇N₄O₅Na₂ (calculated value 709.3342), degree of unsaturation is 18.5.

According to the analytical results of the above spectral data, the final product was in agreement with the chemical structure of 4-(1-hexyloxyethyl)-6,7-bis(propionate sodium)-1,3,5,8-tetramethyl-2-vinylporphyrin.

In the above synthesis method, HVD-1 and HVD-2 were separated firstly, then Photohexer-1 and Photohexer-2 were synthesized respectively. Alternatively, a mixture of 2(4)-(1-hydroxyethyl)-6,7-bis[2-(methoxycarbonyl) ethyl]-1,3,5,8-tetramethyl-4(2)-vinylporphyrin was firstly reacted with 1-hexanol in a solution of dichloromethane saturated by hydrogen bromide gas, then the reaction products were separated and purified by silica gel column chromatography to afford 2-(1-hexyloxyethyl)-6,7-bis[2-(methoxy carbonyl)ethyl]-1,3,5,8-tetramethyl-4-vinylporphyrin and 4-(1-hexyloxyethyl)-6,7-bis[2-(methoxycarbonyl)ethyl]-1,3,5,8-tetramethyl-2-vinylporphyrin, which were then made into sodium salt, respectively.

### Example 3: Activity of the Compounds of the Present Invention as a Photosensitizer

### 1. Comparison of Killing Effects of Photodynamic Therapy Mediated by Photohexer-1, Photohexer-2 on Ex Vivo Human Stomach Cancer SGC7901 Cells, Colorectal Cancer Cells (SW-480, SW-620, CT26, and Caco2), Esophageal Cancer Eca-109 Cells, Breast Cancer Cells (MDA-MB-231, MCF-7 Cells and MCF-7/ADR 1) and Liver Cancer 7721 Cells

### 1.1 Experimental Protocol

The cells in logarithmic growth phase were collected and inoculated on 24-well culture plates. When reaching 70%-80% confluence, cells were divided randomly into the following groups: control (the parallel control group without photosensitizer and irradiation), irradiation (irradiation treatment alone without photosensitizer); (photosensitizer treatment alone without irradiation), photodynamic treatment (treatment by photosensitizer in combination with irradiation). For the photosensitizer alone treatment group and photodynamic treatment group, an appropriate amount of Photohexer-1 (P-1) or Photohexer-2 (P-2) was respectively added away from light to give a final concentration of 0.8, 1.6, 3.2, 6.4 μM. Each group set 3 replicates. Cells were incubated with the active agent, different treatments were carried out according to the grouping. Three different light intensities were selected for the photodynamic treatment group, which were 2.6, 5.2 and 12.4 J/cm², respectively. After treatment and digestion of each group of cells, it was inoculated into a 96-well plate at 100 μL/well, and four wells were set for each treatment. MTT assay was performed after 24 h from inoculation: 20 μL MTT solution was added to each well under the condition away from light, and the culture was continued for 4 hours. 150 μL DMSO was added to each well after discarding the supernatant. After 15 min of dissolution, the absorbance value (OD value) was measured using a microplate reader and the relative survival rate of cells was calculated according to the following formula:

$$Cell relative survival rate = \frac{The OD value of experimental group}{The OD value of control group} \times 100 \%$$

### 1.2 Results

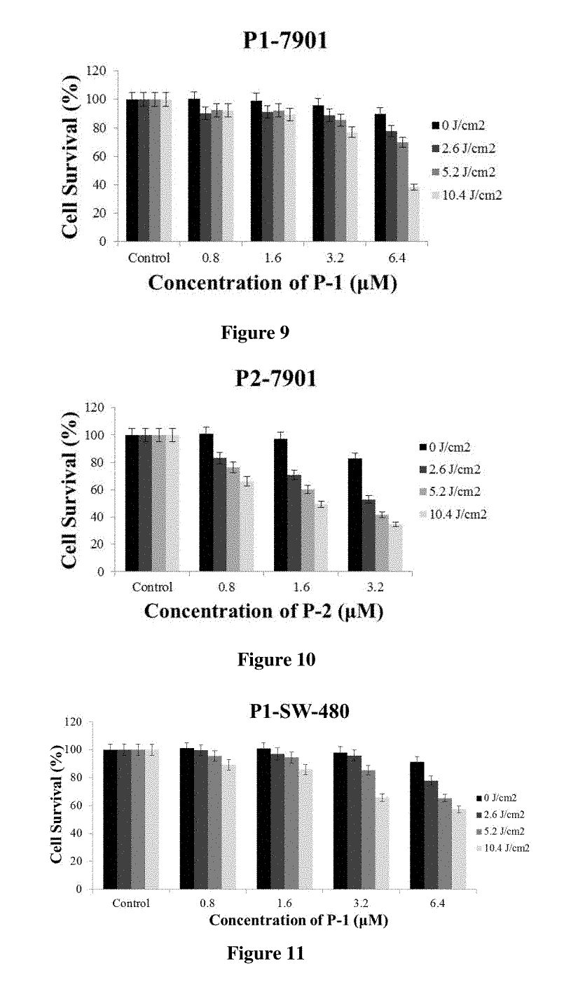

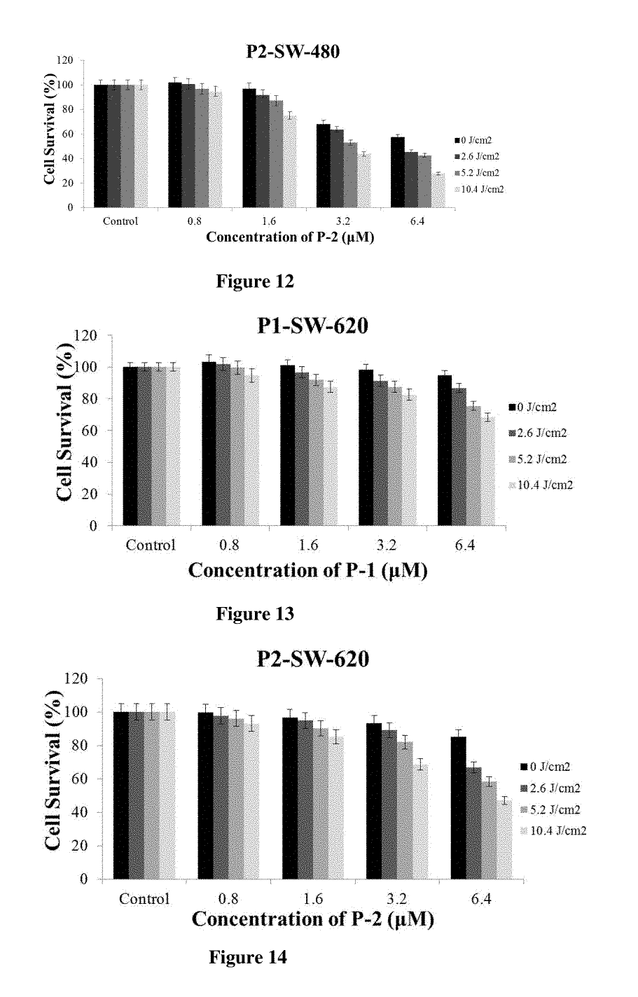

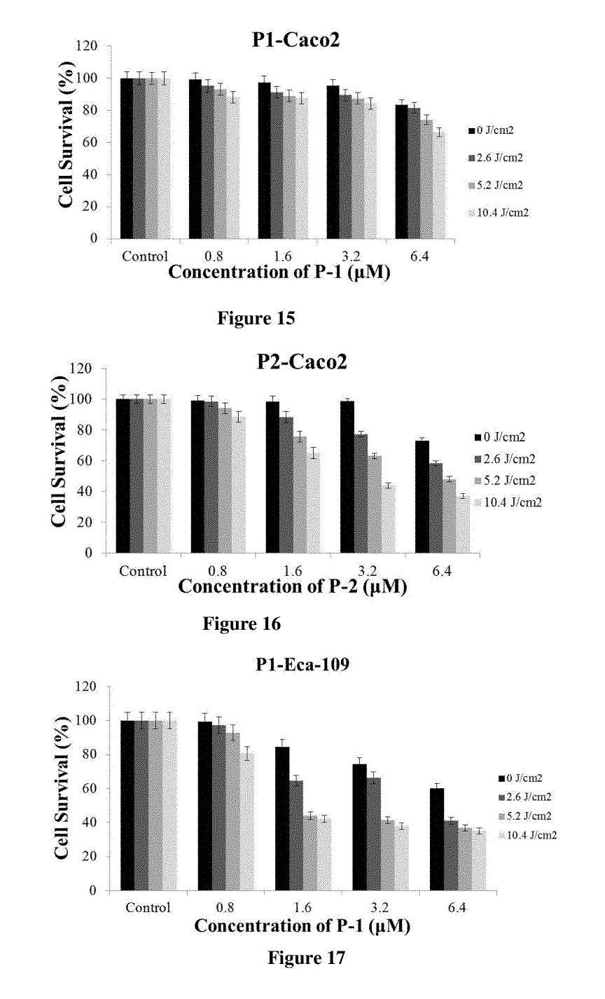

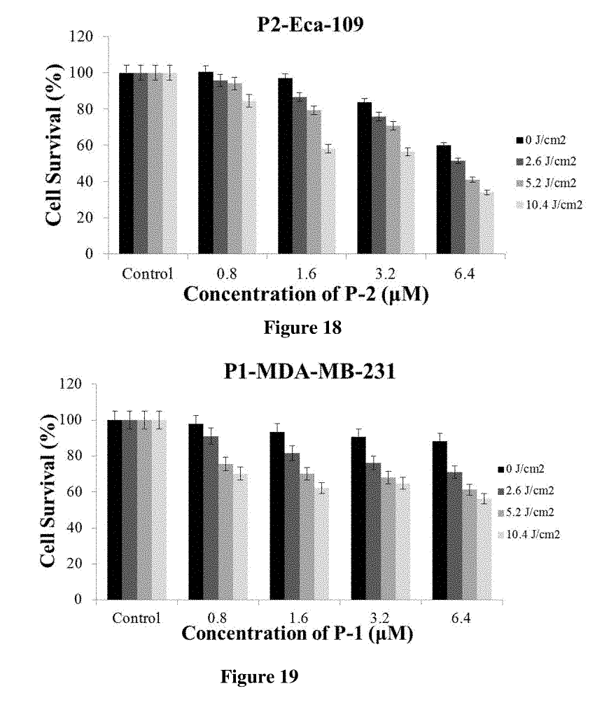

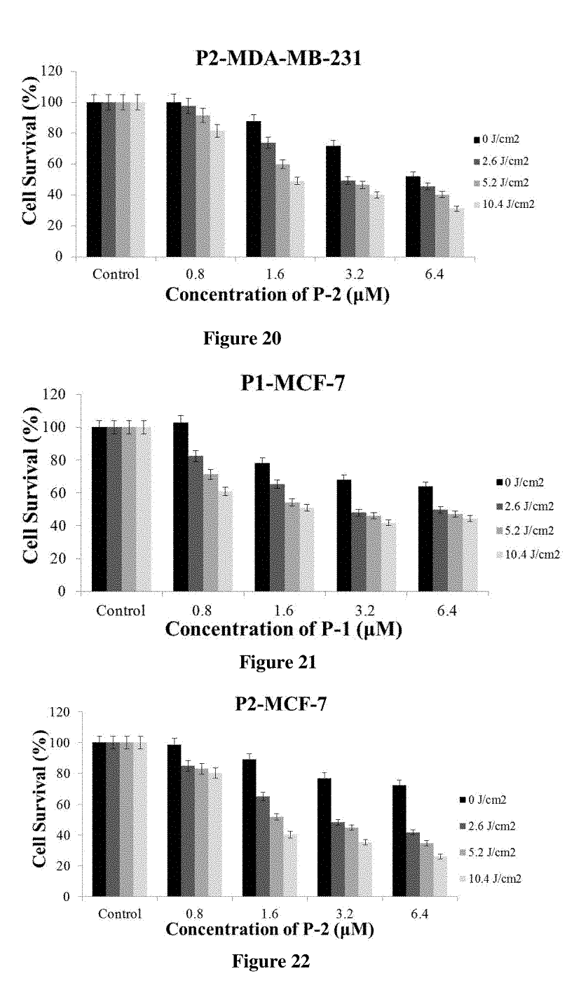

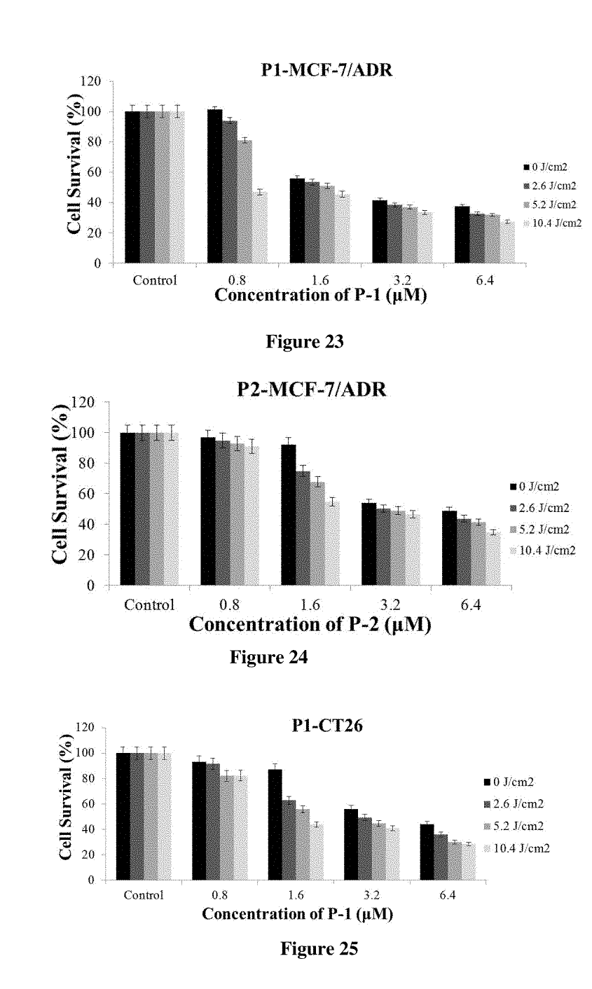

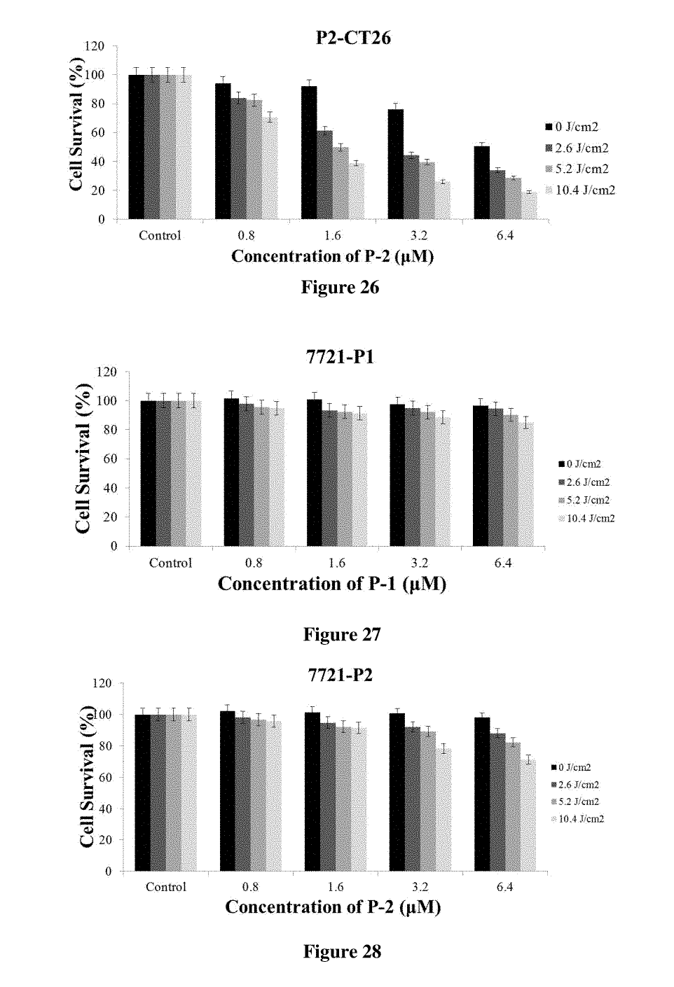

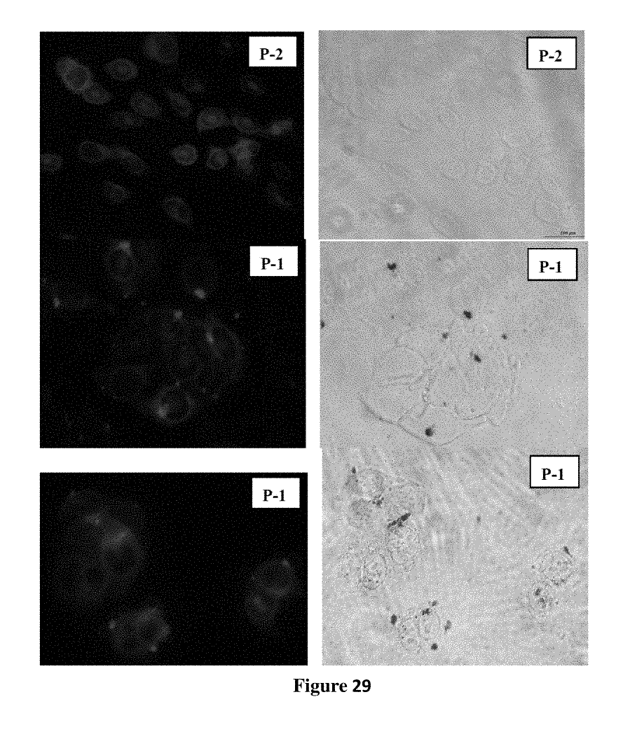

The detailed results were shown in the following Table 1-20 and the FIGS. 9-22:

TABLE 1
The killing effect of photohexer-1 (P-1) mediated
photodynamic therapy on SGC7901 cells.

| Groups | | Sample | Cell survival rate (%) |
|---|---|---|---|
| Control | | 3 | 100.00 ± 2.07 |
| Irradiation | | 3 | 101.89 ± 1.04 |
| P-1 alone treatment (μM) | 0.8 | 3 | 100.39 ± 0.20 |
| | 1.6 | 3 | 99.22 ± 1.37 |
| | 3.2 | 3 | 95.80 ± 1.59 |
| | 6.4 | 3 | 89.77 ± 4.99 |
| P-1 at different | 0.8 | 3 | 90.25 ± 2.16 |
| concentration (μM) | 1.6 | 3 | 91.19 ± 2.89 |
| combined with 2.6 J/cm² | 3.2 | 3 | 88.70 ± 1.24 |
| light irradiation | 6.4 | 3 | 77.76 ± 0.22 |
| P-1 at different | 0.8 | 3 | 92.41 ± 0.63 |
| concentration (μM) | 1.6 | 3 | 92.29 ± 0.15 |
| combined with 5.2 J/cm² | 3.2 | 3 | 85.49 ± 0.99 |
| light irradiation | 6.4 | 3 | 69.94 ± 0.56 |
| P-1 at different | 0.8 | 3 | 92.26 ± 1.16 |
| concentration (μM) | 1.6 | 3 | 89.21 ± 0.24 |
| combined with 10.4 J/cm² | 3.2 | 3 | 77.02 ± 0.04 |
| light irradiation | 6.4 | 3 | 38.34 ± 0.13 |

TABLE 2
The killing effect of photohexer-2(P-2) mediated
photodynamic therapy on SGC7901 cells.

| Groups | | Sample | Cell survival rate (%) |
|---|---|---|---|
| Control | | 3 | 100.00 ± 0.78 |
| Irradiation | | 3 | 97.67 ± 0.99 |
| P-2 alone treatment (μM) | 0.8 | 3 | 100.80 ± 0.91 |
| | 1.6 | 3 | 97.25 ± 1.34 |
| | 3.2 | 3 | 82.72 ± 0.54 |
| P-2 at different | 0.8 | 3 | 83.27 ± 0.82 |
| concentration (μM) | 1.6 | 3 | 70.77 ± 7.19 |
| combined with 2.6 J/cm² | 3.2 | 3 | 52.91 ± 1.96 |
| light irradiation | | | |
| P-2 at different | 0.8 | 3 | 76.47 ± 1.65 |
| concentration (μM) | 1.6 | 3 | 60.00 ± 9.82 |
| combined with 5.2 J/cm² | 3.2 | 3 | 41.72 ± 0.35 |
| light irradiation | | | |
| P-2 at different | 0.8 | 3 | 66.13 ± 0.60 |
| concentration (μM) | 1.6 | 3 | 49.32 ± 12.57 |
| combined with 10.4 J/cm² | 3.2 | 3 | 34.58 ± 0.66 |
| light irradiation | | | |

TABLE 3
The killing effect of photohexer-1 (P-1) mediated
photodynamic therapy on SW-480 cells.

| Groups | | Sample | Cell survival rate (%) |
|---|---|---|---|
| Control | | 3 | 100.00 ± 3.07 |
| Irradiation | | 3 | 100.89 ± 2.04 |
| P-1 alone treatment (μM) | 0.8 | 3 | 101.14 ± 3.20 |
| | 1.6 | 3 | 100.83 ± 2.47 |
| | 3.2 | 3 | 98.02 ± 2.59 |
| | 6.4 | 3 | 91.27 ± 1.09 |
| P-1 at different | 0.8 | 3 | 99.72 ± 3.16 |
| concentration (μM) | 1.6 | 3 | 97.08 ± 2.89 |
| combined with 2.6 J/cm² | 3.2 | 3 | 96.00 ± 2.24 |
| light irradiation | 6.4 | 3 | 77.99 ± 1.22 |
| P-1 at different | 0.8 | 3 | 95.61 ± 3.11 |
| concentration (μM) | 1.6 | 3 | 94.55 ± 2.77 |
| combined with 5.2 J/cm² | 3.2 | 3 | 85.27 ± 1.89 |
| light irradiation | 6.4 | 3 | 65.38 ± 1.32 |
| P-1 at different | 0.8 | 3 | 89.28 ± 3.01 |
| concentration (μM) | 1.6 | 3 | 85.85 ± 2.68 |
| combined with 10.4 J/cm² | 3.2 | 3 | 65.70 ± 2.01 |
| light irradiation | 6.4 | 3 | 57.31 ± 1.03 |

TABLE 4
The killing effect of photohexer-2 (P-2) mediated
photodynamic therapy on SW-480 cells.

| Groups | | Sample | Cell survival rate (%) |
|---|---|---|---|
| Control | | 3 | 100.00 ± 4.07 |
| Irradiation | | 3 | 100.24 ± 3.04 |
| P-2 alone treatment (μM) | 0.8 | 3 | 101.79 ± 4.08 |
| | 1.6 | 3 | 96.97 ± 4.27 |
| | 3.2 | 3 | 68.15 ± 2.78 |
| | 6.4 | 3 | 57.31 ± 2.33 |
| P-2 at different | 0.8 | 3 | 100.79 ± 3.76 |
| concentration (μM) | 1.6 | 3 | 91.72 ± 2.89 |
| combined with 2.6 J/cm² | 3.2 | 3 | 63.40 ± 2.24 |
| light irradiation | 6.4 | 3 | 45.36 ± 1.22 |
| P-2 at different | 0.8 | 3 | 96.70 ± 3.63 |
| concentration (μM) | 1.6 | 3 | 87.06 ± 2.51 |
| combined with 5.2 J/cm² | 3.2 | 3 | 53.01 ± 1.99 |
| light irradiation | 6.4 | 3 | 42.56 ± 1.06 |
| P-2 at different | 0.8 | 3 | 94.70 ± 4.16 |
| concentration (μM) | 1.6 | 3 | 75.11 ± 3.24 |
| combined with 10.4 J/cm² | 3.2 | 3 | 43.68 ± 2.54 |
| light irradiation | 6.4 | 3 | 27.83 ± 1.13 |

TABLE 5
The killing effect of photohexer-1 (P-1) mediated
photodynamic therapy on SW-620 cells.

| Groups | | Sample | Cell survival rate (%) |
|---|---|---|---|
| Control | | 3 | 100.00 ± 4.17 |
| Irradiation | | 3 | 101.88 ± 2.99 |
| P-1 alone treatment (μM) | 0.8 | 3 | 101.33 ± 3.20 |
| | 1.6 | 3 | 100.99 ± 2.47 |
| | 3.2 | 3 | 98.23 ± 1.59 |
| | 6.4 | 3 | 94.83 ± 1.09 |
| P-1 at different | 0.8 | 3 | 100.74 ± 3.46 |
| concentration (μM) | 1.6 | 3 | 96.64 ± 2.79 |
| combined with 2.6 J/cm² | 3.2 | 3 | 91.39 ± 2.18 |
| light irradiation | 6.4 | 3 | 86.70 ± 1.07 |
| P-1 at different | 0.8 | 3 | 99.53 ± 3.71 |
| concentration (μM) | 1.6 | 3 | 91.84 ± 2.97 |
| combined with 5.2 J/cm² | 3.2 | 3 | 87.50 ± 1.69 |
| light irradiation | 6.4 | 3 | 75.53 ± 1.03 |
| P-1 at different | 0.8 | 3 | 94.65 ± 4.04 |
| concentration (μM) | 1.6 | 3 | 87.51 ± 3.16 |
| combined with 10.4 J/cm² | 3.2 | 3 | 82.58 ± 2.31 |
| light irradiation | 6.4 | 3 | 68.33 ± 1.11 |

TABLE 6
The killing effect of photohexer-2 (P-2) mediated
photodynamic therapy on SW-620 cells.

| Groups | | Sample | Cell survival rate (%) |
|---|---|---|---|
| Control | | 3 | 100.00 ± 4.14 |
| Irradiation | | 3 | 98.87 ± 3.57 |
| P-2 alone treatment (μM) | 0.8 | 3 | 101.79 ± 4.27 |
| | 1.6 | 3 | 96.58 ± 3.66 |
| | 3.2 | 3 | 93.16 ± 2.71 |
| | 6.4 | 3 | 85.21 ± 1.03 |
| P-2 at different | 0.8 | 3 | 97.58 ± 3.86 |
| concentration (μM) | 1.6 | 3 | 94.77 ± 2.99 |
| combined with 2.6 J/cm² | 3.2 | 3 | 89.07 ± 2.34 |
| light irradiation | 6.4 | 3 | 66.83 ± 1.12 |
| P-2 at different | 0.8 | 3 | 96.11 ± 4.06 |
| concentration (μM) | 1.6 | 3 | 90.19 ± 3.18 |
| combined with 5.2 J/cm² | 3.2 | 3 | 82.00 ± 2.62 |
| light irradiation | 6.4 | 3 | 58.46 ± 1.23 |
| P-2 at different | 0.8 | 3 | 92.99 ± 3.86 |
| concentration (μM) | 1.6 | 3 | 84.95 ± 3.02 |
| combined with 10.4 J/cm² | 3.2 | 3 | 68.54 ± 2.77 |
| light irradiation | 6.4 | 3 | 47.12 ± 1.04 |

TABLE 7
The killing effect of photohexer-1 (P-1) mediated
photodynamic therapy on Caco2 cells.

| Groups | | Sample | Cell survival rate (%) |
|---|---|---|---|
| Control | | 3 | 100.00 ± 4.37 |
| Irradiation | | 3 | 102.03 ± 3.89 |
| P-1 alone treatment (μM) | 0.8 | 3 | 99.34 ± 3.40 |
| | 1.6 | 3 | 97.36 ± 2.42 |
| | 3.2 | 3 | 95.34 ± 1.67 |
| | 6.4 | 3 | 83.33 ± 1.24 |
| P-1 at different | 0.8 | 3 | 95.31 ± 3.86 |
| concentration (μM) | 1.6 | 3 | 91.33 ± 2.69 |
| combined with 2.6 J/cm² | 3.2 | 3 | 89.49 ± 2.15 |
| light irradiation | 6.4 | 3 | 81.65 ± 1.07 |
| P-1 at different | 0.8 | 3 | 93.10 ± 3.96 |
| concentration (μM) | 1.6 | 3 | 89.01 ± 2.88 |
| combined with 5.2 J/cm² | 3.2 | 3 | 87.46 ± 1.67 |
| light irradiation | 6.4 | 3 | 74.26 ± 1.14 |
| P-1 at different | 0.8 | 3 | 88.18 ± 4.24 |
| concentration (μM) | 1.6 | 3 | 87.63 ± 3.49 |
| combined with 10.4 J/cm ² | 3.2 | 3 | 84.32 ± 2.76 |
| light irradiation | 6.4 | 3 | 66.34 ± 1.17 |

TABLE 8
The killing effect of photohexer-2 (P-2) mediated
photodynamic therapy on Caco2 cells.

| Groups | | Sample | Cell survival rate (%) |
|---|---|---|---|
| Control | | 3 | 100.00 ± 4.64 |
| Irradiation | | 3 | 100.98 ± 3.97 |
| P-2 alone treatment (μM) | 0.8 | 3 | 99.09 ± 4.27 |
| | 1.6 | 3 | 98.50 ± 3.63 |
| | 3.2 | 3 | 94.12 ± 2.54 |
| | 6.4 | 3 | 88.45 ± 1.44 |
| P-2 at different | 0.8 | 3 | 98.47 ± 3.69 |
| concentration (μM) | 1.6 | 3 | 88.31 ± 2.67 |
| combined with 2.6 J/cm² | 3.2 | 3 | 75.55 ± 2.01 |
| light irradiation | 6.4 | 3 | 64.87 ± 1.13 |
| P-2 at different | 0.8 | 3 | 98.57 ± 4.26 |
| concentration (μM) | 1.6 | 3 | 77.26 ± 3.66 |
| combined with 5.2 J/cm² | 3.2 | 3 | 63.08 ± 2.35 |
| light irradiation | 6.4 | 3 | 43.88 ± 1.14 |
| P-2 at different | 0.8 | 3 | 73.07 ± 3.86 |
| concentration (μM) | 1.6 | 3 | 58.33 ± 2.60 |
| combined with 10.4 J/cm² | 3.2 | 3 | 47.94 ± 2.31 |
| light irradiation | 6.4 | 3 | 37.06 ± 1.54 |

TABLE 9
The killing effect of photohexer-1 (P-1) mediated
photodynamic therapy on Eca-109 cells.

| Groups | | Sample | Cell survival rate (%) |
|---|---|---|---|
| Control | | 3 | 100.00 ± 4.78 |
| Irradiation | | 3 | 101.43 ± 4.34 |
| P-1 alone treatment (μM) | 0.8 | 3 | 99.32 ± 3.43 |
| | 1.6 | 3 | 97.28 ± 2.48 |
| | 3.2 | 3 | 92.81 ± 1.37 |
| | 6.4 | 3 | 80.68 ± 1.04 |
| P-1 at different | 0.8 | 3 | 84.64 ± 3.79 |
| concentration (μM) | 1.6 | 3 | 64.63 ± 2.39 |
| combined with 2.6 J/cm² | 3.2 | 3 | 44.02 ± 2.05 |
| light irradiation | 6.4 | 3 | 42.24 ± 1.01 |
| P-1 at different | 0.8 | 3 | 74.34 ± 4.23 |
| concentration (μM) | 1.6 | 3 | 66.40 ± 3.12 |
| combined with 5.2 J/cm² | 3.2 | 3 | 41.43 ± 1.27 |
| light irradiation | 6.4 | 3 | 37.97 ± 1.04 |
| P-1 at different | 0.8 | 3 | 60.09 ± 4.54 |
| concentration (μM) | 1.6 | 3 | 41.05 ± 3.47 |
| combined with 10.4 J/cm² | 3.2 | 3 | 36.89 ± 2.26 |
| light irradiation | 6.4 | 3 | 35.04 ± 1.34 |

TABLE 10
The killing effect of photohexer-2 (P-2) mediated
photodynamic therapy on Eca-109 cells.

| Groups | | Sample | Cell survival rate (%) |
|---|---|---|---|
| Control | | 3 | 100.00 ± 4.43 |
| Irradiation | | 3 | 102.02 ± 4.07 |
| P-2 alone treatment (μM) | 0.8 | 3 | 100.38 ± 4.27 |
| | 1.6 | 3 | 95.76 ± 3.16 |
| | 3.2 | 3 | 94.07 ± 2.58 |
| | 6.4 | 3 | 84.49 ± 1.32 |
| P-2 at different | 0.8 | 3 | 96.96 ± 3.87 |
| concentration (μM) | 1.6 | 3 | 86.66 ± 2.94 |
| combined with 2.6 J/cm² | 3.2 | 3 | 79.33 ± 2.24 |
| light irradiation | 6.4 | 3 | 83.57 ± 1.12 |
| P-2 at different | 0.8 | 3 | 75.83 ± 4.22 |
| concentration (μM) | 1.6 | 3 | 70.66 ± 3.78 |
| combined with 5.2 J/cm² | 3.2 | 3 | 56.40 ± 2.33 |
| light irradiation | 6.4 | 3 | 33.91 ± 1.07 |
| P-2 at different | 0.8 | 3 | 59.89 ± 4.24 |
| concentration (μM) | 1.6 | 3 | 51.36 ± 3.67 |
| combined with 10.4 J/cm² | 3.2 | 3 | 40.90 ± 2.34 |
| light irradiation | 6.4 | 3 | 33.91 ± 1.01 |

TABLE 11
The killing effect of photohexer-1 (P-1) mediated
photodynamic therapy on MDA-MB-231 cells.

| Groups | | Sample | Cell survival rate (%) |
|---|---|---|---|
| Control | | 3 | 100.00 ± 4.82 |
| Irradiation | | 3 | 98.97 ± 4.24 |
| P-1 alone treatment (μM) | 0.8 | 3 | 97.82 ± 3.12 |
| | 1.6 | 3 | 91.07 ± 2.02 |
| | 3.2 | 3 | 75.52 ± 1.07 |
| | 6.4 | 3 | 70.10 ± 0.99 |
| P-1 at different | 0.8 | 3 | 93.29 ± 3.66 |
| concentration (μM) | 1.6 | 3 | 81.63 ± 2.87 |
| combined with 2.6 J/cm² | 3.2 | 3 | 70.24 ± 2.03 |
| light irradiation | 6.4 | 3 | 62.22 ± 1.04 |
| P-1 at different | 0.8 | 3 | 90.67 ± 4.08 |
| concentration (μM) | 1.6 | 3 | 76.33 ± 2.91 |
| combined with 5.2 J/cm² | 3.2 | 3 | 68.08 ± 1.43 |
| light irradiation | 6.4 | 3 | 64.88 ± 1.07 |
| P-1 at different | 0.8 | 3 | 88.23 ± 4.09 |
| concentration (μM) | 1.6 | 3 | 71.13 ± 3.79 |
| combined with 10.4 J/cm² | 3.2 | 3 | 61.29 ± 2.27 |
| light irradiation | 6.4 | 3 | 56.27 ± 1.06 |

TABLE 12
The killing effect of photohexer-2 (P-2) mediated
photodynamic therapy on MDA-MB-231 cells.

| Groups | | Sample | Cell survival rate (%) |
|---|---|---|---|
| Control | | 3 | 100.00 ± 3.87 |
| Irradiation | | 3 | 101.66 ± 4.09 |
| P-2 alone treatment (μM) | 0.8 | 3 | 100.17 ± 4.03 |
| | 1.6 | 3 | 97.69 ± 3.63 |
| | 3.2 | 3 | 91.44 ± 2.57 |
| | 6.4 | 3 | 81.55 ± 1.21 |
| P-2 at different | 0.8 | 3 | 87.82 ± 4.09 |
| concentration (μM) | 1.6 | 3 | 73.80 ± 3.22 |
| combined with 2.6 J/cm² | 3.2 | 3 | 59.92 ± 2.94 |
| light irradiation | 6.4 | 3 | 49.18 ± 1.33 |
| P-2 at different | 0.8 | 3 | 71.8 ± 4.16 |
| concentration (μM) | 1.6 | 3 | 49.35 ± 3.46 |
| combined with 5.2 J/cm² | 3.2 | 3 | 46.57 ± 2.43 |
| light irradiation | 6.4 | 3 | 40.03 ± 1.12 |
| P-2 at different | 0.8 | 3 | 52.23 ± 4.16 |
| concentration (μM) | 1.6 | 3 | 45.69 ± 3.16 |
| combined with 10.4 J/cm² | 3.2 | 3 | 40.31 ± 2.28 |
| light irradiation | 6.4 | 3 | 31.15 ± 0.96 |

TABLE 13
The killing effect of photohexer-1 (P-1) mediated
photodynamic therapy on MCF-7 cells.

| Groups | | Sample | Cell survival rate (%) |
|---|---|---|---|
| Control | | 3 | 100.00 ± 4.83 |
| Irradiation | | 3 | 100.88 ± 4.24 |
| P-1 alone treatment (μM) | 0.8 | 3 | 102.94 ± 3.04 |
| | 1.6 | 3 | 82.48 ± 2.24 |
| | 3.2 | 3 | 71.23 ± 1.76 |
| | 6.4 | 3 | 60.81 ± 1.04 |
| P-1 at different | 0.8 | 3 | 78.02 ± 4.55 |
| concentration (μM) | 1.6 | 3 | 65.32 ± 2.43 |
| combined with 2.6 J/cm² | 3.2 | 3 | 54.13 ± 2.04 |
| light irradiation | 6.4 | 3 | 50.88 ± 1.02 |
| P-1 at different | 0.8 | 3 | 68.03 ± 3.97 |
| concentration (μM) | 1.6 | 3 | 47.92 ± 2.82 |
| combined with 5.2 J/cm² | 3.2 | 3 | 45.96 ± 1.76 |
| light irradiation | 6.4 | 3 | 41.69 ± 1.02 |
| P-1 at different | 0.8 | 3 | 63.99 ± 4.42 |
| concentration (μM) | 1.6 | 3 | 49.71 ± 3.94 |
| combined with 10.4 J/cm² | 3.2 | 3 | 46.98 ± 2.67 |
| light irradiation | 6.4 | 3 | 44.28 ± 1.07 |

TABLE 14
The killing effect of photohexer-2 (P-2) mediated
photodynamic therapy on MCF-7 cells.

| Groups | | Sample | Cell survival rate (%) |
|---|---|---|---|
| Control | | 3 | 100.00 ± 4.65 |
| Irradiation | | 3 | 99.76 ± 3.27 |
| P-2 alone treatment (μM) | 0.8 | 3 | 98.46 ± 4.62 |
| | 1.6 | 3 | 84.93 ± 3.16 |
| | 3.2 | 3 | 82.94 ± 2.28 |
| | 6.4 | 3 | 80.23 ± 1.03 |
| P-2 at different | 0.8 | 3 | 89.08 ± 3.99 |
| concentration (μM) | 1.6 | 3 | 65.11 ± 2.73 |
| combined with 2.6 J/cm² | 3.2 | 3 | 51.79 ± 2.10 |
| light irradiation | 6.4 | 3 | 40.31 ± 1.02 |
| P-2 at different | 0.8 | 3 | 76.83 ± 4.26 |
| concentration (μM) | 1.6 | 3 | 48.15 ± 3.12 |
| combined with 5.2 J/cm² | 3.2 | 3 | 44.80 ± 2.22 |
| light irradiation | 6.4 | 3 | 35.46 ± 1.02 |
| P-2 at different | 0.8 | 3 | 72.40 ± 4.14 |
| concentration (μM) | 1.6 | 3 | 41.81 ± 3.77 |
| combined with 10.4 J/cm² | 3.2 | 3 | 34.81 ± 2.43 |
| light irradiation | 6.4 | 3 | 26.14 ± 1.07 |

TABLE 15
The killing effect of photohexer-1 (P-1) mediated
photodynamic therapy on MCF-7/ADR cells.

| Groups | | Sample | Cell survival rate (%) |
|---|---|---|---|
| Control | | 3 | 100.00 ± 4.86 |
| Irradiation | | 3 | 102.33 ± 4.51 |
| P-1 alone treatment (μM) | 0.8 | 3 | 100.36 ± 3.70 |
| | 1.6 | 3 | 93.95 ± 2.47 |
| | 3.2 | 3 | 81.01 ± 1.58 |
| | 6.4 | 3 | 46.84 ± 1.02 |
| P-1 at different | 0.8 | 3 | 55.89 ± 3.97 |
| concentration (μM) | 1.6 | 3 | 53.43 ± 2.96 |
| combined with 2.6 J/cm² | 3.2 | 3 | 50.99 ± 2.31 |
| light irradiation | 6.4 | 3 | 45.48 ± 1.04 |
| P-1 at different | 0.8 | 3 | 41.47 ± 4.88 |
| concentration (μM) | 1.6 | 3 | 38.42 ± 2.51 |
| combined with 5.2 J/cm² | 3.2 | 3 | 36.95 ± 1.89 |
| light irradiation | 6.4 | 3 | 33.47 ± 1.41 |
| P-1 at different | 0.8 | 3 | 37.45 ± 4.34 |
| concentration (μM) | 1.6 | 3 | 32.58 ± 3.54 |
| combined with 10.4 J/cm² | 3.2 | 3 | 31.79 ± 2.65 |
| light irradiation | 6.4 | 3 | 27.27 ± 1.07 |

TABLE 16
The killing effect of photohexer-2 (P-2) mediated
photodynamic therapy on MCF-7/ADR cells.

| Groups | | Sample | Cell survival rate (%) |
|---|---|---|---|
| Control | | 3 | 100.00 ± 4.67 |
| Irradiation | | 3 | 101.23 ± 3.79 |
| P-2 alone treatment (μM) | 0.8 | 3 | 96.93 ± 4.47 |
| | 1.6 | 3 | 92.26 ± 3.26 |
| | 3.2 | 3 | 53.84 ± 2.58 |
| | 6.4 | 3 | 48.88 ± 1.13 |
| P-2 at different | 0.8 | 3 | 94.88 ± 3.94 |
| concentration (μM) | 1.6 | 3 | 74.79 ± 2.77 |
| combined with 2.6 J/cm² | 3.2 | 3 | 50.37 ± 2.13 |
| light irradiation | 6.4 | 3 | 43.68 ± 1.06 |
| P-2 at different | 0.8 | 3 | 92.76 ± 4.32 |
| concentration (μM) | 1.6 | 3 | 67.82 ± 3.12 |
| combined with 5.2 J/cm² | 3.2 | 3 | 48.99 ± 2.16 |
| light irradiation | 6.4 | 3 | 41.49 ± 0.96 |
| P-2 at different | 0.8 | 3 | 90.99 ± 4.36 |
| concentration (μM) | 1.6 | 3 | 54.66 ± 3.43 |
| combined with 10.4 J/cm² | 3.2 | 3 | 46.57 ± 2.76 |
| light irradiation | 6.4 | 3 | 34.75 ± 1.22 |

TABLE 17
The killing effect of photohexer-1 (P-1) mediated
photodynamic therapy on CT26 cells.

| Groups | | Sample | Cell survival rate (%) |
|---|---|---|---|
| Control | | 3 | 100.00 ± 4.18 |
| Irradiation | | 3 | 97.89 ± 4.24 |
| P-1 alone treatment (μM) | 0.8 | 3 | 93.23 ± 3.43 |
| | 1.6 | 3 | 87.15 ± 2.45 |
| | 3.2 | 3 | 56.13 ± 1.69 |
| | 6.4 | 3 | 44.05 ± 1.26 |
| P-1 at different | 0.8 | 3 | 91.63 ± 3.46 |
| concentration (μM) | 1.6 | 3 | 62.86 ± 2.29 |
| combined with 2.6 J/cm² | 3.2 | 3 | 49.48 ± 2.06 |
| light irradiation | 6.4 | 3 | 36.03 ± 1.17 |
| P-1 at different | 0.8 | 3 | 82.12 ± 4.98 |
| concentration (μM) | 1.6 | 3 | 55.88 ± 2.95 |
| combined with 5.2 J/cm² | 3.2 | 3 | 44.86 ± 1.57 |
| light irradiation | 6.4 | 3 | 30.01 ± 1.07 |
| P-1 at different | 0.8 | 3 | 82.39 ± 4.63 |
| concentration (μM) | 1.6 | 3 | 44.00 ± 3.29 |
| combined with 10.4 J/cm² | 3.2 | 3 | 41.00 ± 2.66 |
| light irradiation | 6.4 | 3 | 28.55 ± 1.47 |

TABLE 18
The killing effect of photohexer-2 (P-2) mediated
photodynamic therapy on CT26 cells.

| Groups | | Sample | Cell survival rate (%) |
|---|---|---|---|
| Control | | 3 | 100.00 ± 4.64 |
| Irradiation | | 3 | 98.89 ± 3.77 |
| P-2 alone treatment (μM) | 0.8 | 3 | 94.08 ± 4.27 |
| | 1.6 | 3 | 92.09 ± 3.56 |
| | 3.2 | 3 | 76.30 ± 2.28 |
| | 6.4 | 3 | 50.49 ± 1.03 |
| P-2 at different | 0.8 | 3 | 83.97 ± 3.96 |
| concentration (μM) | 1.6 | 3 | 61.29 ± 2.78 |
| combined with 2.6 J/cm² | 3.2 | 3 | 44.29 ± 2.41 |
| light irradiation | 6.4 | 3 | 34.11 ± 1.02 |
| P-2 at different | 0.8 | 3 | 82.55 ± 4.77 |
| concentration (μM) | 1.6 | 3 | 49.85 ± 3.55 |
| combined with 5.2 J/cm² | 3.2 | 3 | 39.69 ± 2.27 |
| light irradiation | 6.4 | 3 | 28.61 ± 1.09 |
| P-2 at different | 0.8 | 3 | 70.66 ± 4.66 |
| concentration (μM) | 1.6 | 3 | 38.89 ± 3.86 |
| combined with 10.4 J/cm² | 3.2 | 3 | 26.01 ± 2.38 |
| light irradiation | 6.4 | 3 | 18.82 ± 1.01 |

TABLE 19
The killing effect of photohexer-1 (P-1) mediated
photodynamic therapy on 7721 cells.

| Groups | | Sample | Cell survival rate (%) |
|---|---|---|---|
| Control | | 3 | 100.70 ± 4.45 |
| Irradiation | | 3 | 101.98 ± 3.89 |
| P-1 alone treatment (μM) | 0.8 | 3 | 101.56 ± 4.08 |
| | 1.6 | 3 | 100.75 ± 4.14 |
| | 3.2 | 3 | 97.51 ± 3.98 |
| | 6.4 | 3 | 96.52 ± 3.94 |
| P-1 at different | 0.8 | 3 | 97.88 ± 4.08 |
| concentration (μM) | 1.6 | 3 | 93.31 ± 3.99 |
| combined with 2.6 J/cm² | 3.2 | 3 | 94.92 ± 3.80 |
| light irradiation | 6.4 | 3 | 94.42 ± 3.87 |
| P-1 at different | 0.8 | 3 | 95.52 ± 3.42 |
| concentration (μM) | 1.6 | 3 | 92.36 ± 2.91 |
| combined with 5.2 J/cm² | 3.2 | 3 | 92.13 ± 2.77 |
| light irradiation | 6.4 | 3 | 90.25 ± 4.07 |
| P-1 at different | 0.8 | 3 | 94.75 ± 4.13 |
| concentration (μM) | 1.6 | 3 | 91.35 ± 3.59 |
| combined with 10.4 J/cm² | 3.2 | 3 | 88.59 ± 2.96 |
| light irradiation | 6.4 | 3 | 84.98 ± 1.97 |

TABLE 20
The killing effect of photohexer-2 (P-2) mediated
photodynamic therapy on 7721 cells.

| Groups | | Sample | Cell survival rate (%) |
|---|---|---|---|
| Control | | 3 | 100.20 ± 4.14 |
| Irradiation | | 3 | 101.89 ± 3.47 |
| P-2 alone treatment (μM) | 0.8 | 3 | 102.15 ± 4.07 |
| | 1.6 | 3 | 101.37 ± 3.36 |
| | 3.2 | 3 | 100.64 ± 2.98 |
| | 6.4 | 3 | 98.14 ± 2.53 |
| P-2 at different | 0.8 | 3 | 98.29 ± 4.01 |
| concentration (μM) | 1.6 | 3 | 94.78 ± 3.88 |
| combined with 2.6 J/cm² | 3.2 | 3 | 92.15 ± 3.76 |
| light irradiation | 6.4 | 3 | 88.02 ± 3.59 |
| P-2 at different | 0.8 | 3 | 96.77 ± 3.76 |
| concentration (μM) | 1.6 | 3 | 92.15 ± 3.64 |
| combined with 5.2 J/cm² | 3.2 | 3 | 89.23 ± 3.35 |
| light irradiation | 6.4 | 3 | 82.25 ± 2.79 |
| P-2 at different | 0.8 | 3 | 95.91 ± 3.95 |
| concentration (μM) | 1.6 | 3 | 91.44 ± 3.73 |
| combined with 10.4 J/cm² | 3.2 | 3 | 78.32 ± 3.19 |
| light irradiation | 6.4 | 3 | 71.15 ± 2.91 |

### 1.3 Conclusions

In summary, in the selected drug concentration range, Photohexer-1 (P-1) or Photohexer-2 (P-2) alone treatment (i.e., without light irradiation) did not produce significant cytotoxicity on the cell lines, however, the photodynamic therapies mediated by them have significant selective toxic effect on different cell lines. In human gastric cancer SGC7901 cells, P-1/P-2 photodynamic therapies showed an obvious killing effect in drug-dose and light-intensity dependent manner, and the activity of P-2 was better than P-1. In colorectal cancer cells, the activity of P-2 was also better than P-1, the mediated photodynamic therapy had good killing effect on Caco2 cells and showed high dependence on concentration and light intensity. P-2 showed better activity than P-1 in SW-480 cells. While, for the other three colorectal cancer cells (SW-620, Caco2, CT26), there was no large difference between the two photosensitizers. In human esophageal cancer Eca-109 cells, P-1 and P-2 both exerted the photosensitive activity, and P-1 showed significantly stronger toxicity than that of P-2; for human breast cancer MDA-MB-231 cells, P-2 were more sensitive than P-1; in contrast, for MCF-7 cell, P-1 was more sensitive than P-2; P-1 and P-2, especially P-1, showed significant cell toxicity in MCF-7/ADR cells. Similarly, CT26 murine colorectal cancer cell line was sensitive to P-1 or P-2, respectively. Toxic side effects of P-1 were higher than those of P-2, but the photoactivity of P-2 was stronger than that of P-1, and showed strong dependence on the concentration and light intensity. For human liver cancer 7721 cells, either P-1 alone or P-1 in combination with light irradiation, there was no obvious toxic and side effects. P-2 alone showed no significant toxicity, but after combination with a certain amount of light, showed stronger cell toxicity.

Under the same experimental conditions, the P-2 and P-2 in the cells were observed by fluorescence microscope. It could be inferred that P-2 entered the cell more efficiently than P-1, and relatively more enriched in the cells. P-1 was easy to form aggregates, and distributed in the form of large particles, and mainly localized in the cell membranes.

### 2. Study on the Tumor-Inhibition Effect of P1 and P2-PDT on 4T1 Xenografted Mice

Experimental groups: blank control (2 mice), P-1 alone treatment (4 mg/kg, 8 mice), P-2 alone treatment (2 mg/kg, 8 mice), P-1 treatment (2 mg/kg, 4 mg/kg, 8 mice/group) combined with light irradiations, P-2 treatment (1 mg/kg, 2 mg/kg, 4 mg/kg, 8 mice/group) combined with light irradiations, 66 Balb/c mice in total.

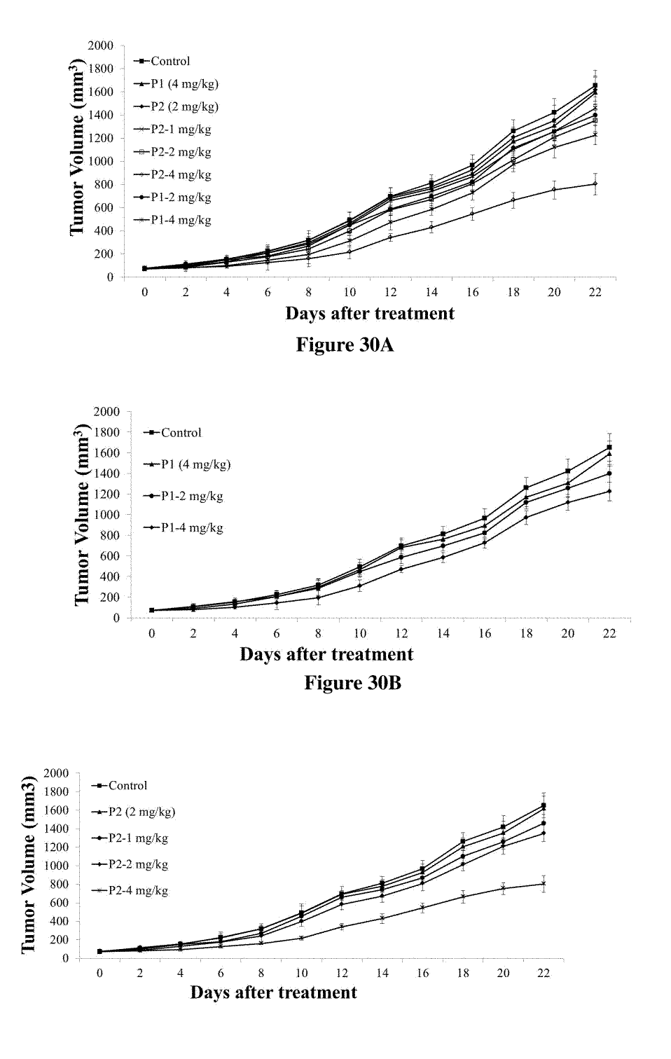

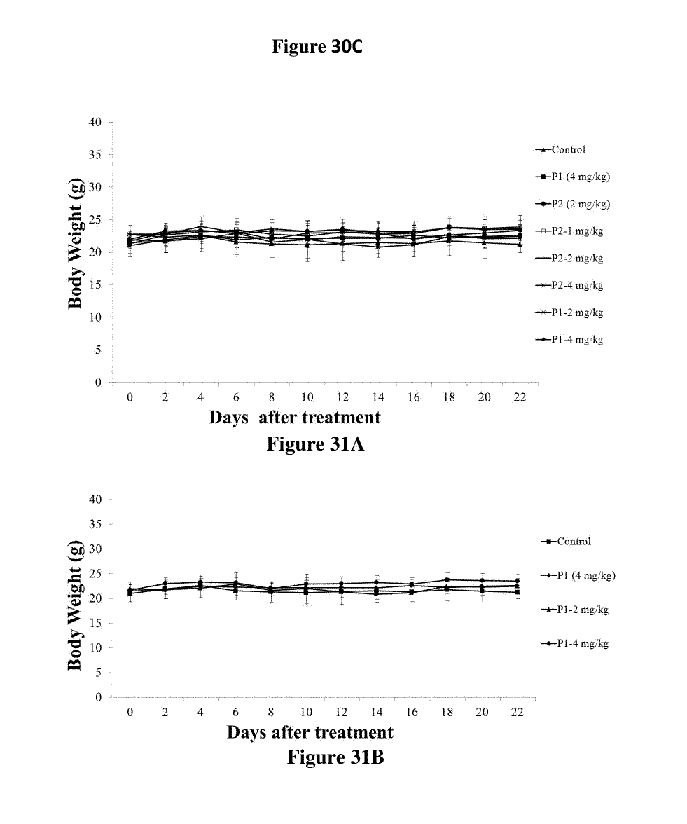

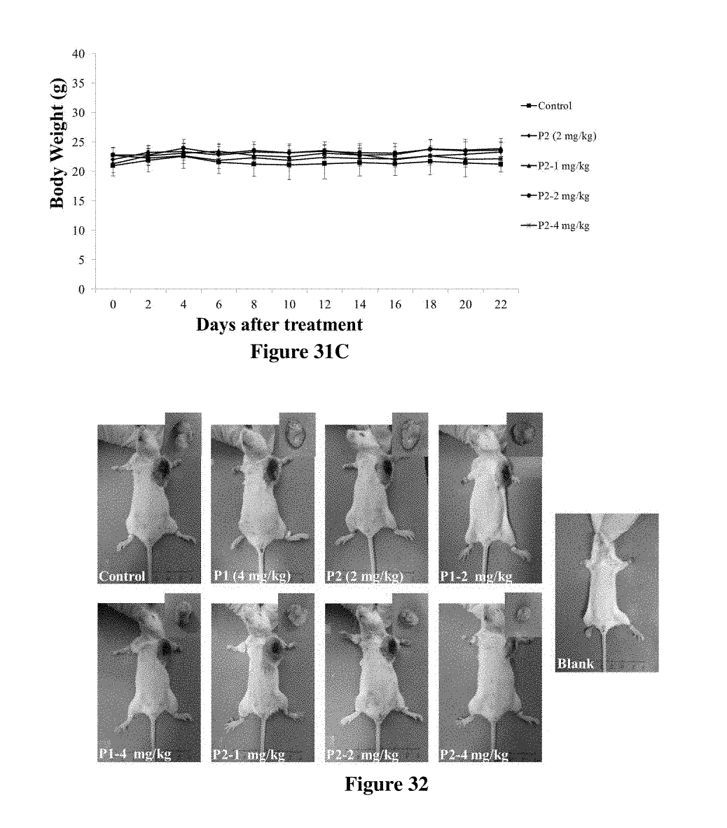

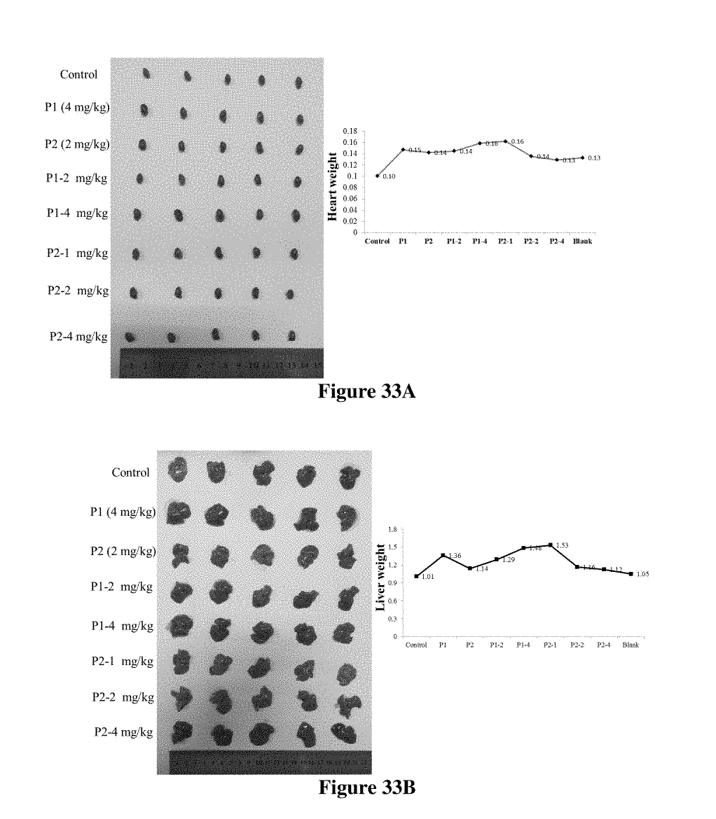

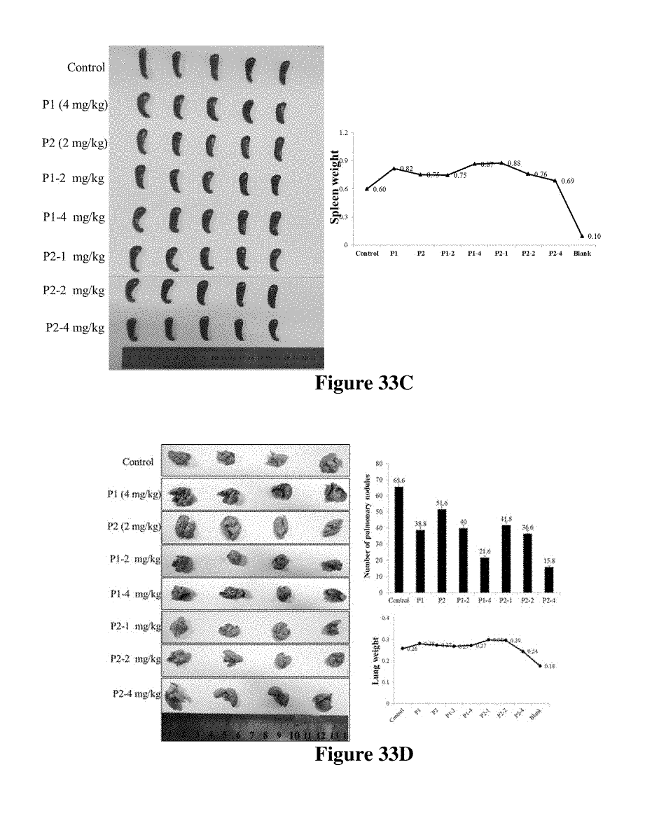

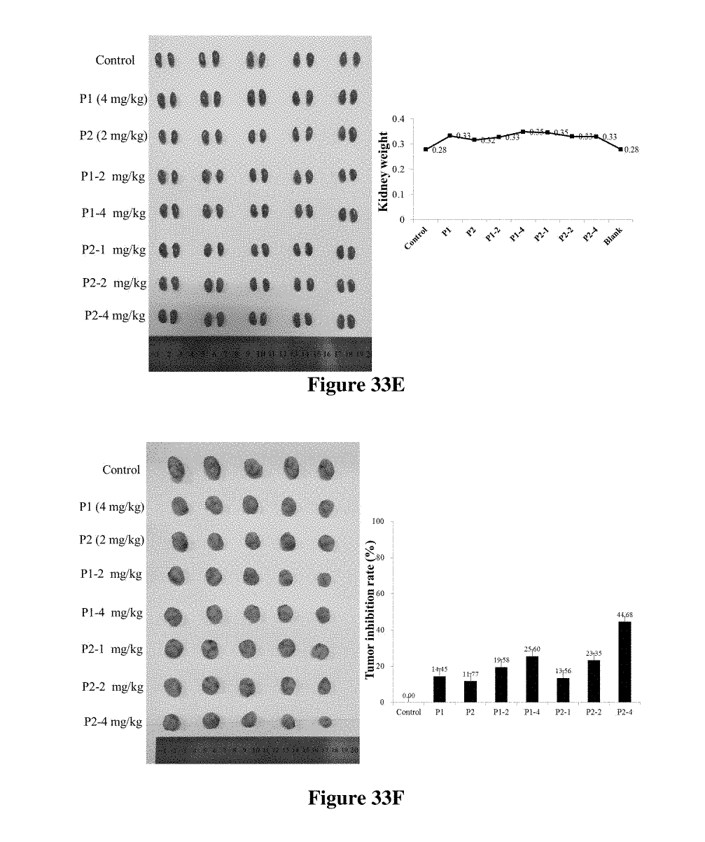

Upon study the inhibitory effect of P1, P2-mediated photodynamic therapy on the growth of 4T1 transplanted tumors, it was found that, among the selected doses of photosensitizers, P1 (4 mg/kg) and P2 (2 mg/kg) alone did not show significant toxic effects. After combination with 50 J/cm² irradiation, P1 and P2 showed obvious dose-dependent activity of photosensitizers, especially P2 (the volume of tumor was maintained at about half the size of tumors of control group on the 8^{th} to 22^{nd} days after treatment). In addition, this treatment also significantly inhibited the increase of tumor weight. On day 22 after treatment, the tumor inhibition rates of P1-4 mg/kg group and P2-2 mg/kg group and P2-4 mg/kg group combined with light irradiation were 25.6%, 23.35% and 44.68%, respectively, which were significantly higher than those of the control group and the drug alone treatment group. After comparison of the body weights of 4T1 tumor-bearing mice in different treatment groups, it was found that various treatment group has no significant effect on body weight of the mice. In addition, compared with the blank control mice, except for significant swelling of the spleen, the other organs did not significant changes. The results are shown in FIGS. 30A to 33F.

Note: FIGS. 30A-33F are all the results on 22 day after treatment;

### Example 4: Solubility Determination

The solubility of Photohexer-1 and Photohexer-2 in water was determined by conventional methods in the art. The results showed that at room temperature, each ml of water dissolved about 3.3 mg Photohexer-1, while each ml of water dissolved about 10 mg Photohexer-2.

### Example 5: Stability Determination

The storage stability of Photohexer-1 and Photohexer-2 was determined by conventional methods in the art. The results showed that there was no content change of Photohexer-1 and Photohexer-2 by HPLC test after 3 months of storage in dark at 2-4.

### Example 6: Lyophilized Powder for Injection

Take a certain amount of Photohexer-1 or Photohexer-2, placed in a light-resistant glass container, add water to dissolve to reach a concentration of 5 mg/ml. The solution is filtered by stainless steel sterilization filter under pressure, firstly through pre-filter membrane with 0.45 μm pore size, then through sterilization membrane with 0.2 μm pore size. The solution is quantitatively dispensed in 10 ml vials in an aseptic operation room, the dispension volume is 2 ml or 1 ml. After lyophilizing under vacuum at −20° C. in a freeze-drying machine, lyophilized powder for injection is obtained.

1. A compound of formula (I) or (II) 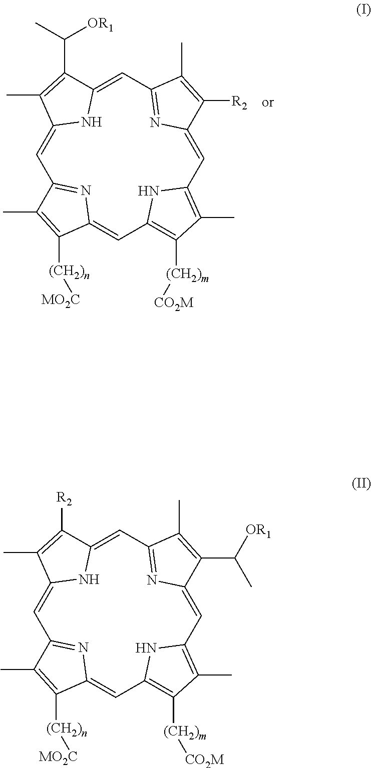
wherein
R₁ is selected from C₁₋₈ alkyl;
R₂ is selected from C₁₋₈ alkyl and C₂₋₈ alkenyl;
M, at each occurrence, is independently selected from alkali metal and alkaline earth metal;
m is an integer selected from 1 to 6; and
n is an integer selected from 1 to 6.

2. The compound of formula (I) or (II) according to claim 1, wherein
R₁ is selected from pentyl, hexyl and heptyl; and
R₂ is selected from ethyl and vinyl.

3. The compound of formula (I) or (II) according to claim wherein
R1 is selected from pentyl, hexyl and heptyl;
R2 is selected from ethyl and vinyl;
M, at each occurrence, is selected independently from sodium and potassium;
m is 2; and
n is 2.

4. The compound of formula (I) or (II) according to claim 1, wherein the two Ms are identical.

5. The compound of formula (I) or (II) according to claim 3, wherein R₁ is n-hexyl.

6. The compound of formula (I) or (II) according to claim 1, selected from the following compounds:

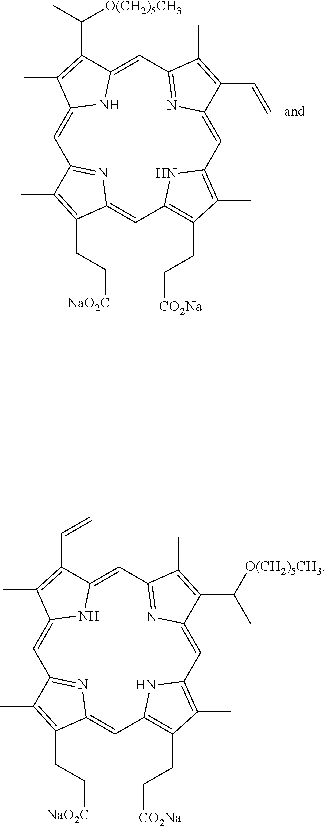

7. A pharmaceutical composition comprising the compound according to claim 1.

8. A method of treating a subject in need thereof, comprising administering to the subject the compound of claim 1, wherein the compound is used as a photosensitizes.

9. A method of treating and/or diagnosing malignant tumors, precancerous lesions or benign lesions in a subject, comprising administering the compound of claim 1 to the subject in a photodynamic therapy.

10. The method of claim 9, wherein the malignant tumors are selected from oral and maxillofacial cancer, nasopharyngeal carcinoma, esophageal cancer, gastric cancer, bile duct cancer, colon cancer, rectal cancer, skin cancer, lung cancer, bronchial carcinoma, breast cancer and subcutaneously metastatic nodules after resection of breast cancer, cervical cancer, liver cancer, bladder cancer, pleural mesothelioma, pancreatic cancer, cancer of the penis, perianal tumor and residual cancer after resection of perianal tumor, Kaposi's sarcoma, prostate cancer, melanoma and brain tumors;
the precancerous lesions are selected from Barrett's esophagus and oral leukoplakia;
the benign lesions are selected from nevus flammeus, age-related macular degeneration, atherosclerosis, rheumatoid arthritis, skin microvascular malformation, psoriasis, and lupus erythematosus skin lesions.

11. A pharmaceutical composition comprising the compound according to claim 3.

12. A pharmaceutical composition comprising the compound according to claim 6.

13. The method of claim 9, wherein
R1 is selected from pentyl, hexyl and heptyl;
R2 is selected from ethyl and vinyl;
M, at each occurrence, is selected independently from sodium and potassium;
m is 2; and
n is 2.

14. The method of claim 9, wherein the compound is [chemical structure]
